(19) United States
(12) Patent Application Publication
(10) Pub. No.: US 2024/0085425 A1
(43) Pub. Date: Mar. 14, 2024

(54) BIOMARKER AND APPLICATION THEREOF

(71) Applicant: ADLAI NORTYE BIOPHARMA CO., LTD., Hangzhou (CN)

(72) Inventors: Nanhai HE, Hangzhou (CN); Youping WANG, Hangzhou (CN); Zhihong LIU, Hangzhou (CN); Yufeng CHEN, Hangzhou (CN)

(73) Assignee: ADLAI NORTYE BIOPHARMA CO., LTD., Hangzhou (CN)

(21) Appl. No.: 18/235,523

(22) Filed: Aug. 18, 2023

(30) Foreign Application Priority Data
Aug. 19, 2022 (CN) 202211002503.3

(51) Int. Cl. G01N 33/574; A61K 31/519; A61P 35/00

(52) U.S. Cl. CPC G01N 33/57484; A61K 31/519; A61P 35/00; G01N 2474/20

(57) ABSTRACT

The present disclosure provides an application of non-phosphorylated β-catenin protein in tumor cells as a biomarker for predicting the responsivity or sensitivity of tumor cells to wnt pathway inhibitors, a method of using non-phosphorylated β-catenin protein in tumor cells as a biomarker to predict the responsivity or sensitivity of tumor cells to wnt pathway inhibitors, a method for treating tumor patients, a method for detecting the content of non-phosphorylated β-catenin in tumor tissues or tumor cells, and a suitable wnt inhibitor.

### CROSS REFERENCE TO RELATED APPLICATIONS

The present application claims the priority of Chinese patent application No. 202211002503.3, filed on Aug. 19, 2022, which is hereby incorporated by reference in its entirety.

### TECHNICAL FIELD

The disclosure belongs to the field of biotechnology and relates to a biomarker, in particular to β-catenin (also known as β-catenin protein or β-catenin) as a biomarker for predicting the responsivity or sensitivity of tumor cells to Wnt/β-catenin pathway inhibitor compound treatment methods and its application.

### BACKGROUND

The Wnt pathway is a basic pathway used to regulate cell proliferation, cell polarity, and control cell fate, and can maintain homeostasis in embryonic and tissue development. In the process of tumorigenesis, β-catenin, a key molecule downstream of Wnt signaling, can accumulate stably in cells, and after entering the nucleus, it can combine with TCF transcription factors to regulate the expression of cell proliferation-related factors and induce abnormal tumor proliferation. During this process, after WNT binds to Frizzled receptor or after, the key molecule of Wnt signal will activate the adjacent LRP5/6 protein, and the pathway enters the “start” state. Next, the protein kinases CKI and GSK3 will bind to the LRP tail. Dishevelled will then also bind to the tails of Frizzled and LRP and phosphorylate Dishevelled. This complex is so stable that it prevents the formation of the regulatory complex that phosphorylates β-catenin. In this way, non-phosphorylated β-catenin (active β-catenin, or active β-catenin) will not be degraded by ubiquitination.

As a key pathway for regulating tumor cell proliferation, the Wnt pathway has been found to exist in a variety of tumors, and its mutation leads to abnormal activation of signaling pathways, thus leading to malignant proliferation of tumor cells. The current Wnt inhibitors have the potential to be used as pan-cancer therapeutic drugs, but the current clinical efficacy of Wnt pathway inhibitors is not good. There are two main reasons. One is that the Wnt pathway inhibitors developed in the early stage mainly target the synthesis and secretion of Wnt ligands, the binding of Wnt ligands and Wnt receptors, and the stable regulation of Wnt receptors. These inhibitors cannot be effective against many cancers with Wnt pathway mutations because they do not depend on the interaction of Wnt ligands and Wnt receptors when these mutant tumor cells proliferate. On the other hand, Wnt pathway inhibitors do not use clear biomarkers for screening patients with abnormal activation of Wnt pathway in the selection of indications, so it is difficult to show clinical effectiveness. Therefore, it is necessary to find a sensitive and specific prediction method for predicting whether tumor cells will respond to Wnt inhibitors.

### BRIEF SUMMARY

In view of the above problems, the present disclosure provides a method of using non-phosphorylated β-catenin (ie active β-catenin) as a sensitive and specific biomarker for predicting whether tumor cells respond to Wnt inhibitors, and the application of the biomarker. The Wnt/β-catenin pathway is a key pathway that regulates tumor cell proliferation. Mutations in a variety of tumors lead to abnormal activation of signaling pathways, thus leading to malignant proliferation of tumor cells. The abnormal blocking of the pathway that regulates the phosphorylation and degradation of β-catenin is one of the keys to the abnormal activation of the wnt signaling pathway, which triggers the continuous and stable accumulation of non-phosphorylated β-catenin (active/active β-catenin) in cells, and non-phosphorylated β-catenin participates in the continuous regulation of transcription into the nucleus, thus leading to abnormal proliferation of tumor cells and aggravating tumor progression.

The contents recorded by the inventor in PCT/CN2021/126539, CN202210114025.9, CN202210115506.1 and CN 202210924700.4 disclose a series of wnt/β-catenin pathway inhibitor compounds or their pharmaceutically acceptable salts, stereoisomers and their preparation processes, which are incorporated herein by reference in their entirety.

In one aspect, the present disclosure provides a use of non-phosphorylated β-catenin protein in tumor cells as a biomarker for predicting the responsivity or sensitivity of said tumor cells to wnt pathway inhibitors.

In one aspect, the present disclosure provides a method of predicting the responsivity or sensitivity of tumor cells to wnt pathway inhibitors, comprising using non-phosphorylated β-catenin protein in said tumor cells as a biomarker. Specifically, the method includes: detecting the content of non-phosphorylated β-catenin protein in tumor cells; comparing the detected value of non-phosphorylated β-catenin protein with a reference value; if the detected value is higher than the reference value, it is considered that the tumor cells are responsive or sensitive to the wnt pathway inhibitor; otherwise, it is considered that the tumor cells are non-responsive or insensitive to the wnt pathway inhibitor.

In one aspect, the present disclosure provides a use of non-phosphorylated β-catenin protein in tumor cells of a tumor patient as a biomarker for predicting the responsivity or sensitivity of the tumor patient to a wnt pathway inhibitor treatment.

In one aspect, the present disclosure provides a method for predicting the responsivity or sensitivity of a tumor patient to a wnt pathway inhibitor treatment, comprising using non-phosphorylated β-catenin protein in tumor cells of the tumor patient as a biomarker. Specifically, the method includes: detecting the content of non-phosphorylated β-catenin protein in the patient's tumor cells; comparing the detected value of the non-phosphorylated β-catenin protein with a reference value; if the detected value is higher than the reference value, it is considered that the tumor cells are responsive or sensitive to the wnt pathway inhibitor treatment method; otherwise, it is considered that the tumor cells are irresponsive or insensitive to the wnt pathway inhibitor treatment method. The method can also include first collecting a sample of the patient's tumor cells.

In one aspect, the present disclosure provides an application of non-phosphorylated β-catenin protein in tumor cells as a biomarker in making a product for predicting the responsivity or sensitivity of the tumor cells to wnt pathway inhibitors, wherein the product can be, for example, a detection kit or a detection solution.

In one aspect, the present disclosure provides a method for treating a tumor patient, comprising: using the above method to predict whether the tumor patient is responsive or sensitive to the wnt pathway inhibitor treatment method; If responsive or sensitive, the wnt pathway inhibitor treatment method is used for the tumor patient.

In one aspect, the present disclosure provides a method for treating a tumor patient in need thereof, comprising: using a Wnt pathway inhibitor treatment method for the tumor patient, wherein the content of non-phosphorylated β-catenin protein in the tumor cells of the patient is higher than the reference value.

In one aspect, the present disclosure provides a method for treating a tumor patient in need thereof, comprising: using a Wnt pathway inhibitor treatment method for the tumor patient, wherein the non-phosphorylated β-catenin protein in the tumor cells of the patient is detected or determined to be higher than the reference value.

In yet another aspect, in the above method of predicting the responsivity or sensitivity of tumor cells or tumor patients to wnt pathway inhibitor treatment method or the method of treating tumor patients, the content of non-phosphorylated β-catenin in tumor cells or in tumor tissues is detected or determined by immunohistochemical (IHC) analysis.

In one aspect, the present disclosure provides a method for detecting the content of non-phosphorylated β-catenin in tumor tissue or tumor cells, comprising performing immunohistochemical (IHC) detection on the tumor cells or tumor cells in the tumor tissue to detect the content of non-phosphorylated β-catenin. Further, the method can be used in the above-mentioned method of predicting the responsivity or sensitivity of tumor cells or tumor patients to wnt pathway inhibitor treatment method or the method of treating tumor patients, so as to detect or determine the content of non-phosphorylated β-catenin in tumor cells.

In one aspect, in the aforementioned applications or methods of the present disclosure, the Wnt pathway inhibitor treatment method comprises administering a therapeutically effective amount of a wnt pathway inhibitor, which may be administered in combination with a second therapeutic agent. Preferably, the second therapeutic agent includes one or more selected from radiotherapy and/or chemotherapy drugs and/or small molecule targeted anti-tumor drugs and/or immunotherapy drugs and/or macromolecular antibody drugs.

In one aspect, in the aforementioned application or method of the present disclosure, the Wnt pathway inhibitor inhibits the activation of Wnt pathway in tumor cells and tumor tissues by reducing the content of non-phosphorylated β-catenin protein in tumor cells and tumor tissues. Preferably, the Wnt pathway inhibitor is a chemical small molecule inhibitor. Further preferably, the wnt pathway inhibitor is selected from the following compounds or pharmaceutically acceptable salts, isotopic derivatives, and stereoisomers thereof:

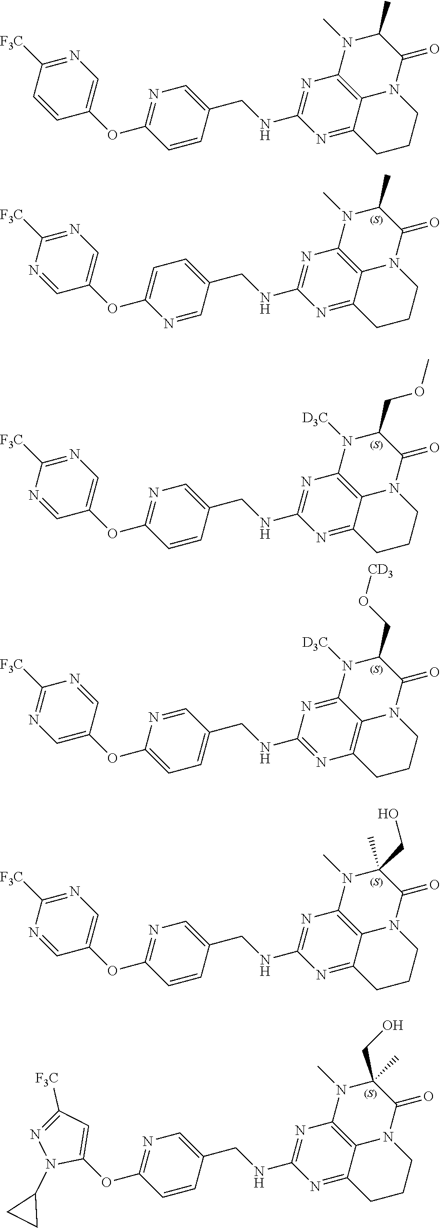

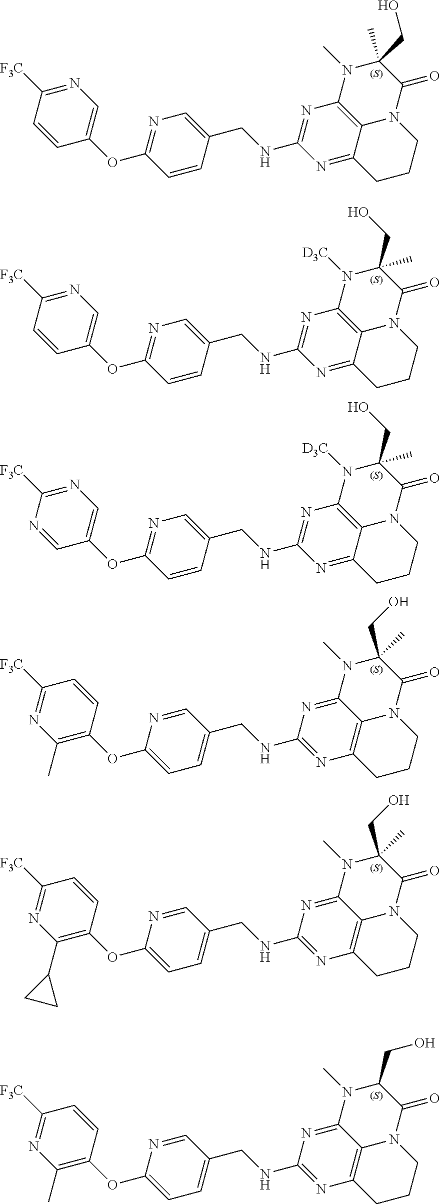

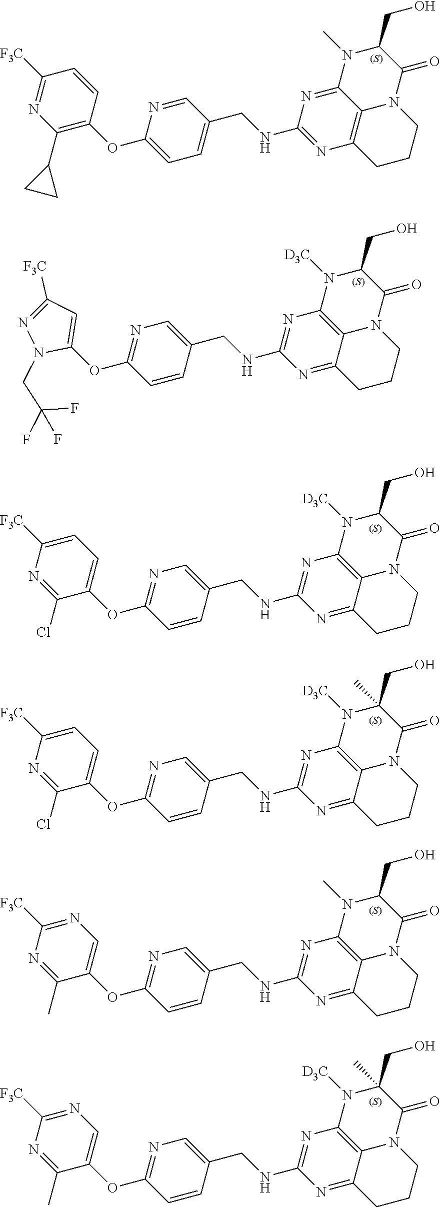

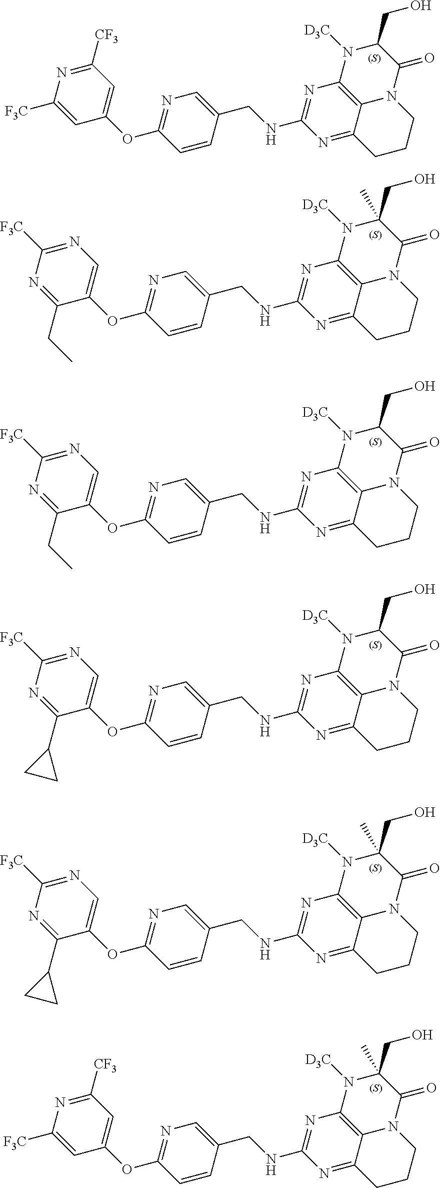

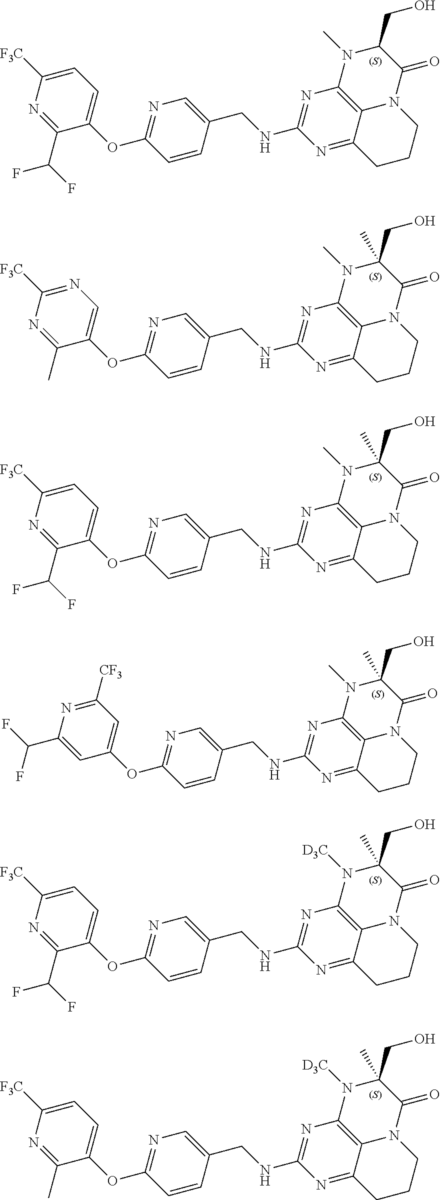

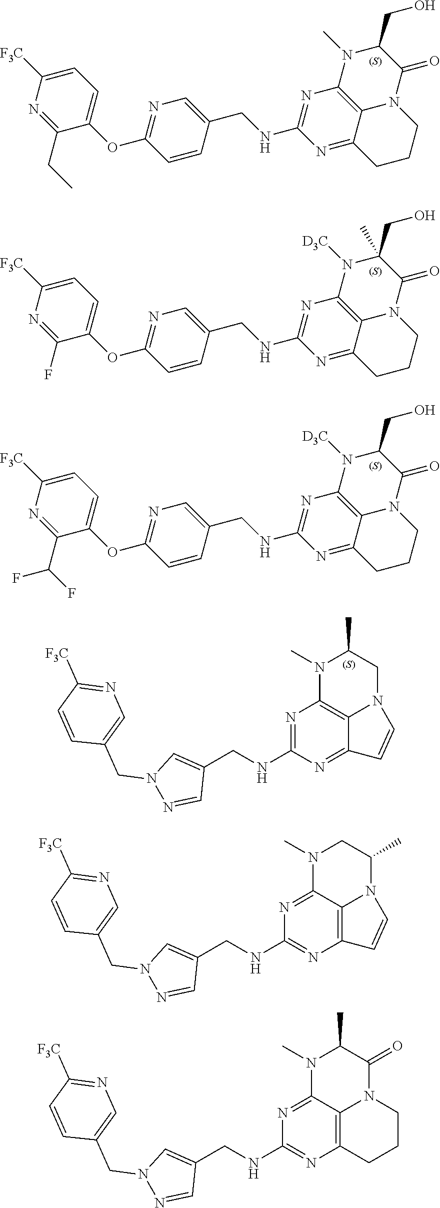

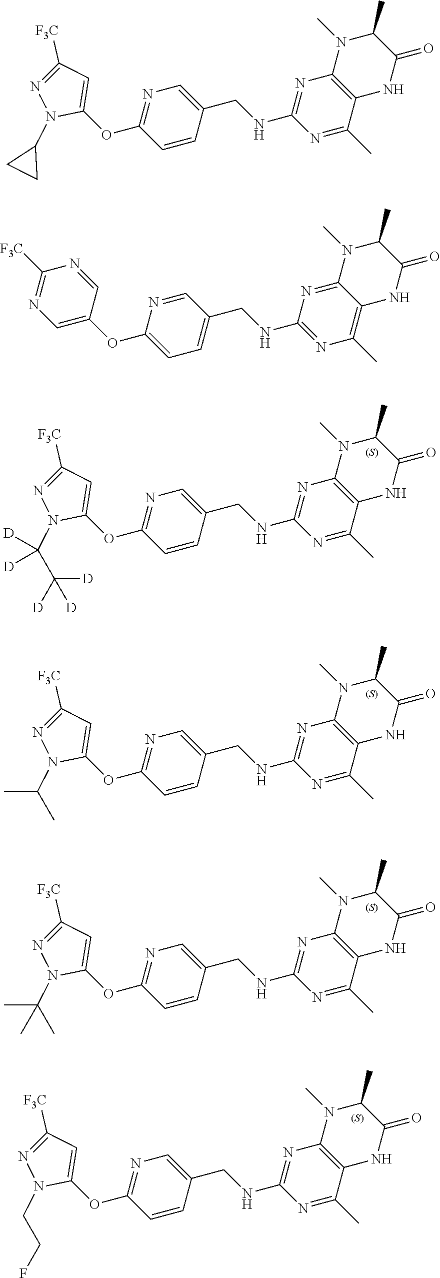

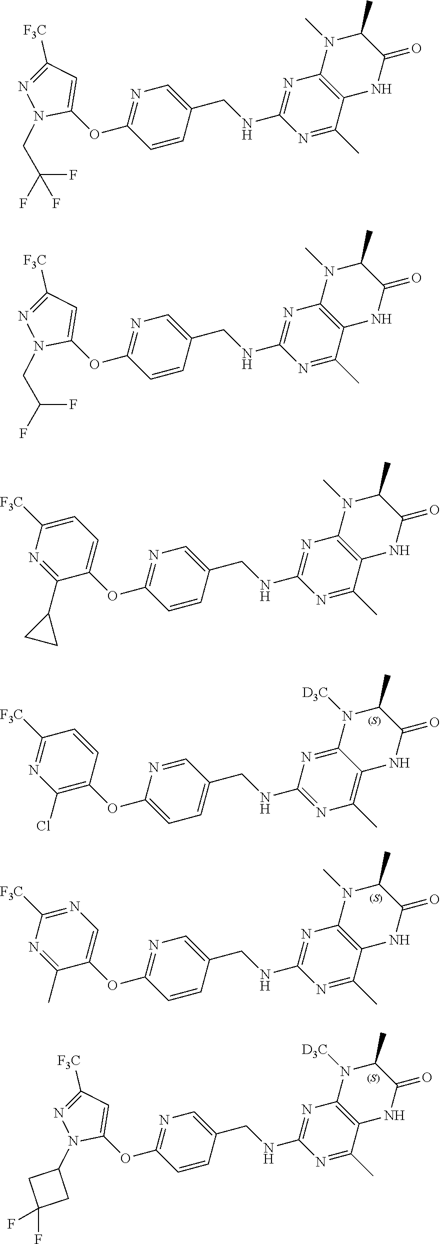

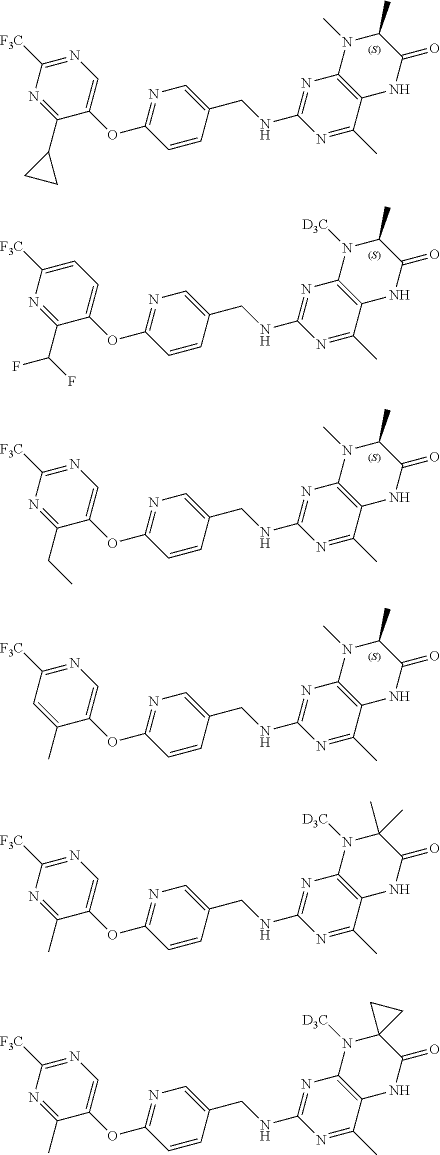

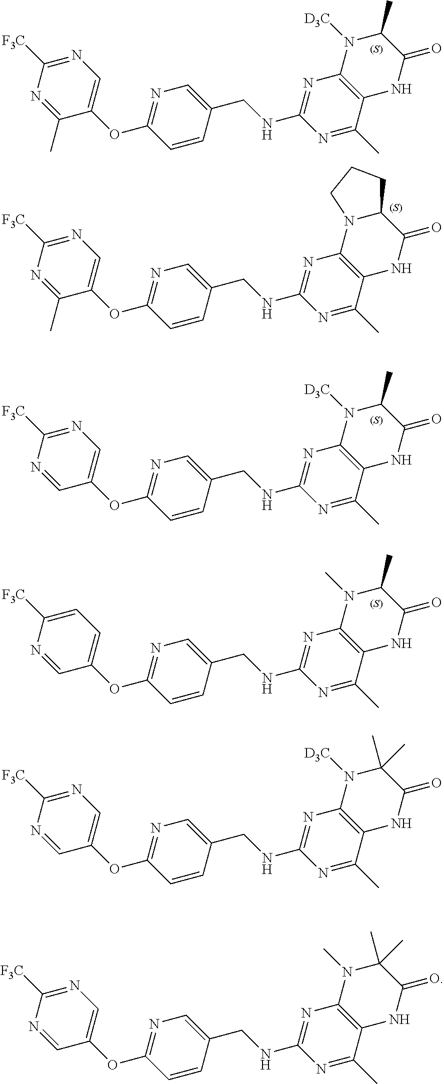

More preferably, the wnt pathway inhibitor is selected from the following compounds or pharmaceutically acceptable salts, isotopic derivatives, and stereoisomers thereof:

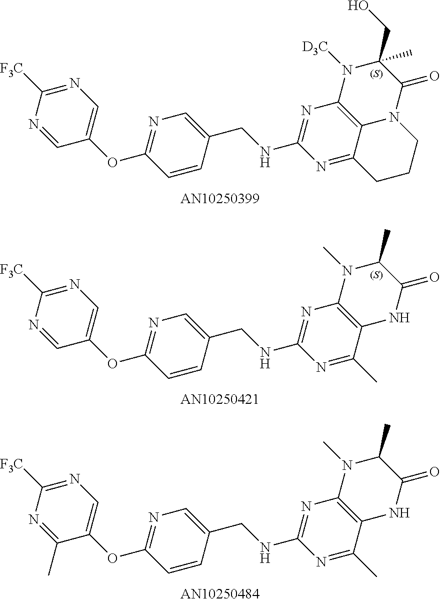

Compared with the prior art, the beneficial effect of the present disclosure is that: the wnt inhibitor that can effectively reduce the content of non-phosphorylated β-catenin has the potential to treat pan-cancer tumors with abnormal activation of the Wnt/β-catenin pathway. The present disclosure finds that non-phosphorylated β-catenin in tumor tissue or tumor cells can be used as a biomarker to screen tumor types and tumor patients who are suitable for this type of wnt inhibitor that can effectively reduce the content of non-phosphorylated β-catenin, rather than just using wnt inhibitors directly as pan-tumor inhibitors, which can effectively shorten the clinical cycle and cost, and at the same time improve the clinical treatment effect and success rate, and may have broad application prospects in clinical practice.

### BRIEF DESCRIPTION OF DRAWINGS

FIG. 1. Compounds AN10250399, AN10250421 and AN10250484 decrease the content of non-phosphorylated β-catenin (active β-catenin) in Colo205 cells.

FIG. 2. MG132 reverses the reduction of the content of non-phosphorylated β-catenin in Colo205 cells and the inhibition towards the transcriptional regulation of TCF/LEF caused by compound AN10250484.

FIG. 3. Comparison of non-phosphorylated β-catenin content in compound-sensitive and non-sensitive tumor cell lines.

FIG. 4. IHC detection of non-phosphorylated β-catenin content in LS174T, Colo205, HepG2 and RKO tumor cell lines.

### DETAILED DESCRIPTION TERMS

The terms used in the present disclosure have meanings commonly understood by those skilled in the art. In order to better understand the present disclosure, some definitions and related abbreviated terms are explained as follows:

The “biomarker” of the present disclosure, also referred to as “biological marker”, refers to a measurable indicator of an individual's biological state. Such biomarkers can be any substances in an individual, as long as they are related to a specific biological state of the individual being tested, such as drug resistance. Such biomarkers may be, but are not limited to, nucleic acid markers, protein markers, cytokine markers, antigen markers, antibody markers, functional markers, and the like. The biomarkers of the present disclosure are specifically protein biomarkers.

The “β-catenin protein” of the present disclosure refers to a functional protein named β-catenin, which is involved in the regulation of cell-cell adhesion and the coordination of gene transcription. In humans, the β-catenin protein is encoded by the CTNNB1 gene (NCBI Gene ID: 1499), and the β-catenin protein acts as a key component of intracellular signal transducers in the Wnt signal transduction pathway. Mutation and overexpression of β-catenin are associated with many cancers, including hepatocellular carcinoma, colorectal cancer, lung cancer, malignant breast tumors, ovarian cancer and endometrial cancer, etc.

“Non-phosphorylated β-catenin” or “active β-catenin” or “active β-catenin” in the present disclosure refers to the non-phosphorylated activated form of β-catenin protein, and there are two states of phosphorylated and non-phosphorylated β-catenin proteins in cells, wherein the generation of phosphorylated β-catenin is triggered by the phosphorylation modification of Ser45 of β-catenin by creatine kinase 1 (CK1). After Ser45 is phosphorylated, β-catenin can be phosphorylated by glycogen synthase kinase 3 (GSK-3). GSK-3β forms a complete phosphorylation state by phosphorylating β-catenin Ser33, Ser37 and Thr41, and phosphorylated β-catenin can be degraded by ubiquitination to maintain a low level of β-catenin state; non-phosphorylated β-catenin is regulated by the activity inhibition of GSK-3β and CK1α, and by immobilizing Axin protein, it destroys the formation of multi-protein complexes, eventually leading to the accumulation of non-phosphorylated β-catenin in cells. After entering the nucleus, aggregated β-catenin forms a complex with TCF to initiate the transcription of downstream target genes.

The “Wnt pathway mutation” of the present disclosure refers to the generation of: the gene mutation of the signal molecule of Wnt pathway leads to excessive transcription mediated by β-catenin, thus activating the gene program that promotes self-renewal, proliferation and survival, mainly through key mutation gene drive pathways. In the typical model of WNT pathway activation in tumors, the mutation of β-catenin destruction complex is an important part of Wnt pathway mutation, which involves the inactivation mutation of APC, AXIN1 and AXIN2 or the activation mutation of CTNNB1 itself. The dysregulation mutations of WNT receptor content include RNF43 and ZNRF3 deletion mutations, which lead to reduced endocytic elimination of WNT receptors and thus enrich a large number of WNT receptors and enhance the sensitivity of ligands binding. In colorectal cancer, APC mutations accumulated the largest proportion in sporadic tumors, reaching 67%, followed by RNF43 (8%), CTNNB1 (6%), and AXIN2 (5%). On the contrary, the main mutation of liver cancer occurs in CTNNB1 gene (25%), followed by AXIN1 (8%) mutation, adrenocortical carcinoma has a mutation ratio of ZNRF3 (20%) or CTNNB1 (15%), and pancreatic cancer tends to enrich mutations in RNF43 (6%).

“Treating” refers to alleviating, inhibiting and/or reversing the progression of a disease (e.g., tumor/cancer) in a subject in need thereof. The term “treatment” includes any indication of successful treatment or amelioration of a disease, including any objective or subjective parameter, such as alleviation; palliation; reduction of symptoms or making the injury, pathology or condition more tolerant to the subject; delay or slowing of the rate of progression, and the like. Measures of treatment or improvement may be based, for example, on the results of physical, pathological, and/or diagnostic tests known in the art. Treating can also refer to reducing the incidence or risk of onset of a disease, or reducing a disease recurrence (e.g., prolonging the time to recurrence), compared to what would occur if the measure were not taken. In the medical field, this treatment is also known as “prophylaxis”.

The term “patient” refers to a mammalian subject, and especially a human subject, including male or female subjects, and includes neonatal, infant, infant, adolescent, adult, or elderly subjects, and further includes various races and ethnicities, such as Caucasian, African, and Asian.

The term “pharmaceutically acceptable salt” refers to a relatively non-toxic, inorganic or organic acid salt of a compound of the present disclosure. These salts can be prepared in situ during the final isolation and purification of the compounds, or by reacting the purified compound in free form with a suitable organic or inorganic acid, respectively, and isolating the salt thus formed. Representative acid salts include (but are not limited to) acetate, adipate, aspartate, benzoate, benzenesulfonate, bicarbonate/carbonate, bisulfate/sulfate, borate, D-camphorsulfonate, citrate, cyclamate, ethanedisulfonate, ethanesulfonate, formate, fumarate, glucoheptonate, gluconic acid salt, glucuronate, hexafluorophosphate, seabenzoate, hydrochloride/chloride, hydrobromide/bromide, hydroiodide/iodide, isethionate, lactic acid Salt, malate, maleate, malonate, methanesulfonate, methylsulfate, naphthylate, 2-naphthalenesulfonate, nicotinate, nitrate, orotic acid Salt, oxalate, palmitate, pamoate, phosphate/hydrogen phosphate/dihydrogen phosphate, pyroglutamate, sugar salt, stearate, succinate, tannin salt, tartrate, tosylate, trifluoroacetate and xinafoate. In one embodiment, the pharmaceutically acceptable salt is a hydrochloride/chloride salt.

Herein, the term “pharmaceutically acceptable” are used interchangeably to refer to the type generally accepted by those skilled in the pharmaceutical art. For example, pharmaceutically acceptable salts, pharmaceutically acceptable carriers and the like.

The term “effective amount” or “therapeutically effective amount” refers to an amount effective to treat a disease, as documented by clinical testing and evaluation, patient observation, and the like. An “effective amount” can further mean an amount that causes a detectable change in biological or chemical activity. A detectable change can be detected and/or further quantified by one skilled in the art familiar with the relevant mechanism or method. Additionally, an “effective amount” can mean an amount that maintains a desired physiological state (i.e., reduces or prevents significant decline and/or promotes amelioration of a condition). An “effective amount” can further refer to a therapeutically effective amount.

“Radiotherapy” as used herein, also known as “radiation therapy”, refers to the medical use of ionizing radiation, especially for the treatment of cancer. Preferably, the medical use of ionizing radiation in cancer treatment results in the reduction and/or killing of cancer cells in the subject. Radiation therapy can be administered by any means known to those skilled in the art. Examples of radiation utilized in radiation therapy include, but are not limited to, photon radiation, ionizing radiation, or charged particle radiation, such as X-rays or protons. Examples of radiation therapy include, but are not limited to: external beam radiation therapy or teletherapy; brachytherapy or sealed beam source therapy; and whole body radioisotope therapy or unsealed source radiation therapy.

The “chemotherapy” of the present disclosure is also called “chemotherapy”, which refers to the use of chemotherapeutic agents to treat cancer subjects, wherein the chemotherapeutic agents include so far common chemotherapeutic agents used for cancer patients, such as taxane, paclitaxel, docetaxel, cabazitaxel, gemcitabine, carboplatin, cisplatin, oxaliplatin, fluorouracil, capecitabine, or tegafor (tegafor), or any functional analog thereof.

The “small molecule targeted anti-tumor drugs” of the present disclosure include, but are not limited to, protein degradation agents, protein stabilizers, protein structure blockers, protein kinase agonists, and protein kinase inhibitors. Wherein, protein kinase inhibitors include but not limited to tyrosine kinase inhibitors, serine and/or threonine kinase inhibitors, poly ADP ribose polymerase (PARP, poly ADP-ribose polymerase) inhibitors. The targets of the inhibitors include but are not limited to Fascin-1 protein, HDAC (histone deacetylase), Proteasome, CD38, SLAMF7 (CS1/CD319/CRACC), RANKL, EGFR (epidermal growth factor receptor), anaplastic lymphoma (ALK), MET gene, ROS1 gene, HER2 gene, RET gene, BRAF gene, PI3K signaling pathway, DDR2 (discoidin death receptor 2) gene, FGFR1 (fibroblast growth factor receptor 1), NTRK1 (neurotrophic tyrosine kinase receptor type 1) gene, KRAS gene. The targets of the small-molecule targeted anti-tumor drugs also include COX-2 (epoxygenase-2), APE1 (apurine apyrimidinic endonuclease), VEGFR (vascular endothelial growth factor receptor), CXCR-4 (chemokine receptor-4), MMP (matrix metalloproteinase), IGF-1R (insulin-like growth factor receptor), Ezrin, PEDF (pigment epithelium-derived factor), AS, ES, OPG (osteoprotegerin), Src, IFN, ALCAM (activated leukocyte cell adhesion molecule), HSP, JIP1, GSK-3 (glycogen synthesis kinase 3 sugar), CyclinD1 (cell cycle regulatory protein), CDK4 (cyclin-dependent kinase), TIMP1 (tissue inhibitor of metalloproteinase), THBS3, PTHR1 (parathyroid hormone-related protein receptor 1), TEM7 (human tumor vascular endothelial marker 7), COPS3, cathepsin K.

“Immunotherapy drug” refers to a drug that activates or regulates the human immune system to achieve a therapeutic effect.

“Macromolecular antibody drug” in the present disclosure refers to a binding protein with at least one antigen-binding domain. Antibodies and fragments thereof of the present application may be whole antibodies or any fragments thereof. Accordingly, the antibodies and fragments of the present application include monoclonal antibodies or fragments thereof and antibody variants or fragments thereof, as well as immunoconjugates. Examples of antibody fragments include Fab fragments, Fab′ fragments, F(ab)′ fragments, Fv fragments, isolated CDR regions, single chain Fv molecules (scFv), and other antibody fragments known in the art. Antibodies and fragments thereof may also include recombinant polypeptides, fusion proteins and bispecific antibodies. The antibodies and fragments thereof disclosed herein may be of the IgG1, IgG2, IgG3 or IgG4 isotype.

So that the disclosure described herein may be more fully understood, the following examples are set forth. It should be understood that these examples are for illustrative purposes only and are not to be construed as limiting the disclosure in any way.

### SPECIFIC EXAMPLES

The present disclosure will be described in further detail below in conjunction with the accompanying drawings and examples. The following examples are only used to illustrate the disclosure and are not intended to limit the scope of the disclosure.

### Biological Screening and Results of Wnt Pathway Inhibitors

### Test Example 1: Compound Sensitive Cell Screening Test

The cell lines used in the experiment are various tumor cells, including DU4475, Colo205, HepG2, PLC/PRF/5, BT-20, LS174T, NCI-H929, HCT116, SW620, SK-CO-1, SW480, DLD1, HT-29, SW900, SW1417, HuH7, H2009, PANC-1, SW48, Duadi, H358, HCT15, 22Rv1, Hela, RKO, Raji, Jurkat, NCI-H716, MDA-MB-231, NCI-H157 and THP1, and the inhibitory effect of the compound of the present disclosure on the proliferation of these tumor cells is determined, in order to screen out sensitive cell types of the compound of the present disclosure.

DU4475, Colo205, HepG2, PLC/PRF/5, BT-20, LS174T, NCI-H929, HCT116, SW620, SK-CO-1, SW480, DLD1, HT-29, SW900, SW1417, HuH7, H2009, PANC-1, SW48, Duadi, H358, HCT15, 22Rv1, Hela, RKO, Raji, Jurkat, NCI-H716, MDA-MB-231, NCI-H157 and THP1 cell lines cultured in each medium were treated during the logarithmic growth phase, and the cells were collected and prepared into a uniform cell suspension of known concentration, and then added to the 96-well cell culture plate to make each well contain 1000 cells. Put it into a 5% CO₂ incubator and incubate at 37° C. for 20-24 h. The next day, the fully dissolved 3-fold gradient diluted compound was added to each cell culture well, so that the final maximum concentration in the cell culture well was 20 μM, and the culture was continued for 72 hours. In this test, Promega's cell viability detection test is used for detection. The more the cells proliferate, the stronger the final signal intensity will be. The detection instrument is SpectraMax, full wavelength mode. The wells with only DMSO added were used as positive control wells, and the wells without cells inoculated were used as negative control wells. The IC₅₀ value of each compound for different cell proliferation inhibition was calculated to evaluate the inhibitory effect of the compound on different tumor cells. The results are shown in Table 1 below.

TABLE 1
The IC₅₀ values of compounds on the proliferation
inhibition of different tumor cell lines

Anti-proliferation IC₅₀ (nM)

| Tumor cell line | AN10250399 | AN10250484 |
|---|---|---|
| DU4475 | 36.94 | 7.9 |
| Colo205 | 43 | 9 |
| HepG2 | 128 | 11 |
| PLC/PRF/5 | 62.64 | 18 |
| BT-20 | 135 | 22 |
| LS174T | 102 | 26.26 |
| NCI-H929 | 396 | 129 |
| HCT116 | 240 | 142 |
| SW620 | 100 | 284 |
| SK-CO-1 | 341 | 285 |
| SW480 | 300 | 300 |
| DLD1 | 96.57 | 315 |
| HT-29 | 131 | 352 |
| SW900 | 57.2 | 26.6 |
| SW1417 | 170 | 33.4 |
| HuH7 | 105.64 | 61.26 |
| H2009 | 213.5 | 91.72 |
| PANC-1 | 140.37 | 47.54 |
| SW48 | >10000 | >10000 |
| Duadi | >10000 | >10000 |
| H358 | >10000 | >10000 |
| HCT15 | >10000 | >10000 |
| 22Rv1 | >10000 | >10000 |
| Hela | >10000 | >10000 |
| RKO | >10000 | >10000 |
| Raji | >10000 | >10000 |
| Jurkat | >10000 | >10000 |
| NCI-H716 | >10000 | >10000 |
| MDA-MB-231 | >10000 | >10000 |
| NCI-H157 | >10000 | >10000 |
| THP1 | >10000 | >10000 |

The results in Table 1 indicate that, first, not all tumor cell lines respond to the wnt inhibitor compounds of the present disclosure. After screening, the present disclosure determines that DU4475, Colo205, HepG2, PLC/PRF/5, BT-20, LS174T, NCI-H929, HCT116, SW620, SK-CO-1, SW480, DLD1, HT-29, SW900, SW1417, HuH7, H2009 and PANC-1 all have more effective responses to the wnt inhibitor compound of the present disclosure and are sensitive cells to wnt inhibitor compounds of the disclosure.

### Test Example 2: Compounds AN10250399, AN10250421 and AN10250484 Reduce the Content of Non-Phosphorylated β-catenin in Sensitive Cells

The Wnt signaling pathway is a key pathway for abnormal tumor growth. In colon cancer, liver cancer, pancreatic cancer and other cancers, a large number of patients have abnormal activation of the Wnt signaling pathway. The Wnt signaling pathway depends on the fine balance of β-catenin protein level. When Wnt signaling is not activated, β-catenin is phosphorylated by CK1 and APC/Axin/GSK-3β complexes, and the β-catenin protein is kept at a low level through ubiquitin-dependent proteasome degradation. When the Wnt signaling pathway is activated, β-catenin maintains a stable state of non-phosphorylation, and non-phosphorylated β-catenin can be transported into the nucleus through Rac1 and other factors, and plays a transcriptional regulatory role by binding to LEF/TCF transcription factors in the nucleus, activates downstream target genes such as C-myc and Cyclin D1, and promotes cell proliferation. Therefore, abnormal activation of the Wnt signaling pathway in tumor cells will lead to abnormal proliferation of tumors. In order to explore the inhibitory mechanism of the compound on the proliferation of tumor cell lines, the compound AN10250399, AN10250421 or AN10250484 was used to treat sensitive cells Colo205, DU4475 and HepG2, and Western blot was used to detect the content of non-phosphorylated β-catenin in the Wnt pathway to clarify the effect of the compound on content of the non-phosphorylated β-catenin.

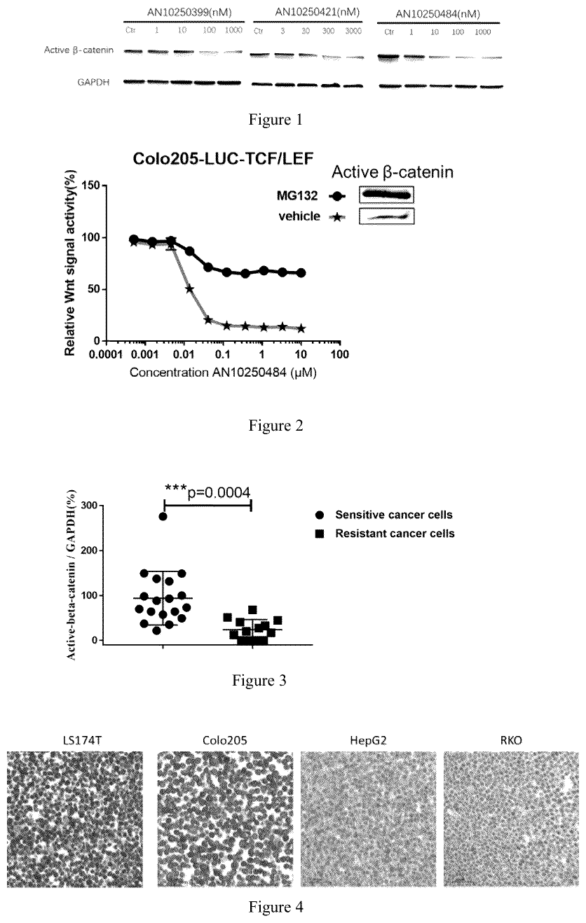

Add 500 μL of the compound with the highest concentration shown in FIG. 1 to each well of the 6-well cell culture plate, and make a 10-fold gradient dilution of the compound concentration. Then 500 μL of 10⁶ Colo205, DU4475 and HepG2 or 500 μL of medium were inoculated into each well, and corresponding solvent treatment was performed simultaneously as negative control wells. The cells were placed in a 5% CO₂ cell incubator, cultured overnight at 37° C. The culture medium was removed, washed once with PBS, 200 μL of RIPA buffer containing protease inhibitors was added to each well, and the protein lysate was collected after lysing on ice for 20 min. Add an appropriate amount of concentrated SDS-PAGE protein loading buffer to the collected protein lysate samples, and heat at 100° C. or in a boiling water bath for 3-5 minutes to fully denature the protein. Load the protein sample directly to the SDS-PAGE gel for electrophoresis separation, transfer the protein on the SDS-PAGE gel to the PVDF membrane; after the transfer was completed, the protein was blocked by Western blocking solution, then Rabbit anti-β-catenin non-phospho S37/After T41 (ab278503) was added and the protein was incubated at room temperature for one hour and washed, the content of non-phosphorylated β-catenin was detected by incubation with Goat Anti-Rabbit IgG H&L (HRP) (ab6721). The results are shown in FIG. 1 and Table 2. As a control, glyceraldehyde-3-phosphate dehydrogenase (GAPDH) was used as an internal standard protein

TABLE 2
IC₅₀ values of compounds for inhibition of non-phosphorylated
β-catenin content in different cell lines

| | Non-phosphorylated β-catenin content inhibition IC50 (nM) | | |
|---|---|---|---|
| cancer cells | AN10250399 | AN10250421 | AN10250484 |
| DU4475 | 157 | 2.2 | 29 |
| Colo205 | 22 | 10 | 10 |
| HepG2 | 4 | 51.6 | 5.9 |

The results in FIG. 1 and Table 2 show that the wnt inhibitor compound of the present disclosure can significantly reduce the content of non-phosphorylated β-catenin (non-phosphorylated β-catenin) in Colo205, DU4475 and HepG2 cells. By reducing the content of non-phosphorylated β-catenin in cells, it can reduce the transcriptional regulation effect of its entry into the nucleus and bind to LEF/TCF transcription factors, thereby slowing down the promotion of cell proliferation, or inhibiting cell proliferation.

### Test Example 3: The Compound Regulates the Activation of Wnt Pathway by Promoting the Degradation of β-catenin in Sensitive Cells

In Test Example 2, the compound of the present disclosure was tested to inhibit the content of non-phosphorylated β-catenin in representative sensitive cells Colo205, DU4475 and HepG2. Non-phosphorylated β-catenin can be transported into the nucleus, and binds to LEF/TCF transcription factors in the nucleus to play a transcriptional regulatory role; in the wnt pathway, non-phosphorylated β-catenin can be converted into phosphorylated β-catenin, while phosphorylated β-catenin is regulated by the ubiquitin-dependent proteasomal degradation system. In order to prove whether the reduction of the content of non-phosphorylated β-catenin by the compound of the present disclosure is associated with the proteasome degradation system, a proteasome inhibitor is used to block the degradation of non-phosphorylated β-catenin to explore whether the reduction of non-phosphorylated β-catenin content by the compound of the present disclosure can be blocked by proteasome inhibitors and affect the transcriptional activity of LEF/TCF.

The Colo205-LUC-TCF/LEF cell line is a reporter tool cell stably transfected with the pGL4.49-LUC2-TCF/LEF vector, and its Wnt/β-catenin pathway is continuously activated. After adding inhibitor, the expression of firefly luciferase regulated by the TCF/LEF cis-element decreased, and after adding the detection substrate, the detected light signal decreased accordingly, thus the inhibitory effect of the compound was detected.

Add 100 μL of the compound AN10250484 at a maximum concentration of 20 μM to each well of the 96-well cell culture plate, and make a 3-fold gradient dilution of the compound concentration. Then inoculate each well with 10,000 stable Colo205-LUC-TCF/LEF cells containing 2 μM MG132 (Proteasome inhibitor) (MCE: HY-13259) or 10,000 Colo205-LUC-TCF/LEF cells containing control solvents, which were treated accordingly as positive and negative control wells. Place the cells in a 5% CO₂ cell incubator and incubate at 37° C. for 4 hours. After 4 hours, remove the culture medium, add 100 μL of reagent (Promega) containing the corresponding firefly luciferase substrate to each well, and measure the luciferase reporter gene activity. Use SpectraMax to read the luminescence intensity in full-wavelength mode. The results are shown in FIG. 2. The compound has the transcriptional activity of inhibiting Wnt pathway TCF/LEF, and the inhibitory effect of the compound on the transcription of TCF/LEF is significantly reduced after adding MG132, which proves that the compound regulation of the transcriptional activity of the Wnt pathway TCF/LEF is associated with the proteasomal degradation system.

Add 500 ul medium containing 200 nM compound plus 2 μM MG132 or 200 nM compound plus control solvent to each well of a 6-well cell culture plate, then add 500 ul containing 10⁶ Colo205-LUC-TCF/LEF cells respectively, and incubate for 4 hours Finally, the protein was collected to detect the content of non-phosphorylated β-catenin. As shown in FIG. 2, the compound of the present disclosure can maintain the level of non-phosphorylated β-catenin in cells, and MG132 can block the effect of the compound of the present disclosure on non-phosphorylated β-catenin. The effect of low-level maintenance of β-catenin is consistent with MG132 reversing the inhibition of Wnt pathway TCF/LEF transcriptional activity by the compound of the present disclosure, and non-phosphorylated β-catenin is a key factor involved in TCF/LEF transcription, so the inventive compound inhibits TCF/LEF transcriptional activity by reducing the content of non-phosphorylated β-catenin.

### Test Example 4: Determination of the Content of Non-Phosphorylated β-catenin in Compound-Sensitive and Non-Sensitive Tumor Cells

In Test Examples 2 and 3, it was tested that the compound of the present disclosure can inhibit the content of non-phosphorylated β-catenin in representative sensitive cells Colo205, DU4475 and HepG2, and affect the activation of Wnt pathway by reducing non-phosphorylated β-catenin. The wnt pathway is a key pathway for tumor cell proliferation. In order to determine whether there is a correlation between the content of non-phosphorylated β-catenin and the inhibitory effect of the compound of the present disclosure on tumor proliferation, the present disclosure analyzed non-phosphorylated β-catenin in sensitive cells and non-sensitive tumor cells. The content of catenin is used to judge whether there is a difference in the content of non-phosphorylated β-catenin in these two types of cells.

DU4475, Colo205, HepG2, PLC/PRF/5, BT-20, LS174T, NCI-H929, HCT116, SW620, SK-CO-1, SW480, DLD1, HT-29, SW900, SW1417, HuH7, H2009, PANC-1, SW48, Duadi, H358, HCT15, 22Rv1, Hela, RKO, Raji, Jurkat, NCI-H716, MDA-MB-231, NCI-H157 and THP1 cell lines cultured in each culture medium was treated in logarithmic growth phase. After the cells were collected and prepared into a uniform cell suspension of known concentration, and then 10⁶ of the above-mentioned cells were inoculated into each well, and the cells were placed in a 5% CO₂ cell incubator, cultured overnight at 37° C., and the protein lysate was collected. The content of non-phosphorylated β-catenin was analyzed, and the results are shown in Table 3 and FIG. 3. The contents of non-phosphorylated β-catenin in sensitive tumor cells and non-sensitive tumor cells were listed, and the results are shown in Table 3. The average content of non-phosphorylated β-catenin in sensitive tumor cells was 94.05% (define the content of non-phosphorylated β-catenin of Colo205, that is, the value of non-phosphorylated β-catenin compared to the internal standard protein GAPDH to be 100%), while the average content of non-sensitive tumor cells was 24.09%, and sensitive tumor cells were significantly higher than non-sensitive tumor cells. FIG. 3 shows that tumor cell lines with relatively high non-phosphorylated β-catenin content are more sensitive to the compound of the present disclosure than cell lines with low non-phosphorylated β-catenin content. The test results show that the level of non-phosphorylated β-catenin in tumor cells can be used to predict whether the cells are sensitive to the compounds of the present disclosure, especially compounds AN1025399, AN1025421 and/or AN1025484. If the content of non-phosphorylated β-catenin in a tumor cell is higher than a certain reference value, it can be judged that the tumor cell is sensitive to the compound of the present disclosure; otherwise, it is not sensitive. Wherein, “reference value” refers to a specific value that is compared with the detected value to determine whether the tumor cell or tumor patient is responsive or sensitive to the wnt inhibitor, which can be obtained, for example, through clinical statistical analysis.

LS174T, Colo205, HepG2 and RKO cells were selected for immunohistochemical (IHC) analysis of non-phosphorylated β-catenin content. After collecting 10⁷ cells, 2 ml of 10% neutral formalin buffer solution was used to fix the cells. Fixation can be carried out at room temperature (15-25° C.), and the fixation time is about 2 hours, then use 300 g for 5 minutes to centrifuge, then pour off the supernatant, and then use 5 ml of 75% alcohol to resuspend the cells. After cell paraffin embedding, dewaxing, antigen repair and blocking, add non-phospho (Active) β-catenin (Ser33/37/Thr41) (CST: D13A1) antibody incubation, use HRP-labeled goat anti-rabbit secondary antibody working solution to incubate, then use DAB/H2O2 reaction staining, and finally use a microscope to take pictures and record after hematoxylin counterstaining. The results are shown in FIG. 4. The IHC staining results show that the content of non-phosphorylated β-catenin was LS174T>Colo205>HepG2>RKO, which was consistent with the results in Table 3.

TABLE 3
Determination of non-phosphorylated β-catenin content
in sensitive and nonsensitive tumor cell lines

| Sensitive cancer cells | | Insensitive cancer cells | |
|---|---|---|---|
| | Relative non- | | Relative non- |
| | phosphorylated | | phosphorylated |
| Cell lines | β-catenin levels | Cell lines | β-catenin levels |
| DU4475 | 98% | SW48 | 51.36% |
| Colo205 | 100.00% | Duadi | 0.0% |
| HepG2 | 57.20% | H358 | 68.20% |
| PLC/PRF/5 | 37.25% | HCT15 | 32.40% |
| BT-20 | 49% | 22Rv1 | 12.00% |
| LS174T | 275.80% | Hela | 27% |
| NCI-H929 | 35% | RKO | 0.00% |
| HCT116 | 92.92% | Raji | 0.00% |
| SW620 | 131.46% | Jurkat | 0.00% |
| SK-CO-1 | 137.25% | NCI-H716 | 20% |
| SW480 | 88.40% | MDA-MB- | 40.47% |
| | | 231 | |
| DLD1 | 148.75% | NCI-H157 | 44.8% |
| HT-29 | 84.00% | THP1 | 17% |
| SW900 | 21.7% | | |
| SW1417 | 69.65% | | |
| HuH7 | 149.28% | | |
| H2009 | 73.14% | | |
| PANC-1 | 64.11% | | |
| Average | 94.05% ± 14.07% | Average | 24.09% ± 6.201% |

Further, the result of the present disclosure also provides a method for tumor treatment. If the content of non-phosphorylated β-catenin in the tumor cells of a patient suffering from a certain cancer is higher than a certain reference value, it can be judged that the patient is sensitive to the treatment of the compound of the present disclosure, so that the patient can be administered a therapeutically effective amount of the compound of the present disclosure, especially compounds AN1025399, AN1025421 and/or AN1025484.

The structures and synthetic methods of compounds AN1025399, AN1025421 and AN1025484 are described in the patents PCT/CN2021/126539, CN202210114025.9, CN202210115506.1 and CN 202210924700.4 previously submitted by the inventor. According to examples of the disclosure, it can be reasonably expected that other compounds with a wnt/β-catenin pathway inhibition mechanism similar to compounds AN1025399, AN1025421, and AN1025484, especially other compounds that have similar wnt/β-catenin pathway inhibitory mechanisms described in the above-mentioned previous patent documents are also applicable to the present disclosure.

1. A method for predicting the responsivity or sensitivity of tumor cells to Wnt pathway inhibitors, comprising using non-phosphorylated β-catenin protein in said tumor cells as a biomarker.

2. The method according to claim 1, comprising: detecting the content of non-phosphorylated β-catenin protein in tumor cells; comparing the detected value of non-phosphorylated β-catenin protein with a reference value; if the detected value is higher than the reference value, it is considered that the tumor cells are responsive or sensitive to the Wnt pathway inhibitor; otherwise, the tumor cells are considered non-responsive or insensitive to the Wnt pathway inhibitor.

3. A method for predicting the responsivity or sensitivity of a tumor patient to a Wnt pathway inhibitor treatment method, comprising using non-phosphorylated β-catenin protein tumor cells of the tumor patient as a biomarker, and wherein the Wnt pathway inhibitor treatment method comprises administering a therapeutically effective amount of a Wnt pathway inhibitor, which may be administered in combination with a second therapeutic agent.

4. The method according to claim 3, comprising: detecting the content the non-phosphorylated β-catenin protein in the tumor cells of the patient; comparing the detected value of the non-phosphorylated β-catenin protein with a reference value; if the detected value is higher than the reference value, it is considered that the tumor cells are responsive or sensitive to the treatment method of the Wnt pathway inhibitor; otherwise, it is considered that the tumor cells are irresponsive or insensitive to the Wnt pathway inhibitor treatment method.

5. A method for treating a tumor patient, comprising: predicting whether the tumor patient is responsive or sensitive to the Wnt pathway inhibitor treatment according to claim 4; if there is a responsivity or sensitivity, then using the Wnt pathway inhibitor treatment on the tumor patient.

6. A method for treating a tumor patient in need thereof, comprising: treating the tumor patient with a Wnt pathway inhibitor, wherein the content of non-phosphorylated β-catenin protein in tumor cells of the patient is higher, or is detected or determined to be higher than a reference value.

7. The method for predicting the responsivity or sensitivity of tumor cells to Wnt pathway inhibitors according to claim 2, wherein the content of non-phosphorylated β-catenin in tumor cells or tumor cells of tumor tissues is detected or determined by immunohistochemistry (IHC) analysis.

8. The method for predicting the responsivity or sensitivity of a tumor patient to Wnt pathway inhibitor treatment method according to claim 4, wherein the content of non-phosphorylated β-catenin in tumor cells or tumor cells of tumor tissues is detected or determined by immunohistochemistry (IHC) analysis.

9. The method for treating a tumor patient according to claim 5, wherein the content of non-phosphorylated β-catenin in tumor cells or tumor cells of tumor tissues is detected or determined by immunohistochemistry (IHC) analysis.

10. The method according to claim 1, wherein the Wnt pathway inhibitor is a chemical small molecule inhibitor.

11. The method according to claim 2, wherein the Wnt pathway inhibitor is a chemical small molecule inhibitor.

12. The method according to claim 3, wherein the Wnt pathway inhibitor is a chemical small molecule inhibitor.

13. The method according to claim 4, wherein the Wnt pathway inhibitor is a chemical small molecule inhibitor.

14. The method according to claim 5, wherein the Wnt pathway inhibitor is a chemical small molecule inhibitor.

15. The method according to claim 6, wherein the Wnt pathway inhibitor is a chemical small molecule inhibitor.

16. The method according to claim 1, wherein the Wnt pathway inhibitor is selected from the following compounds or pharmaceutically acceptable salts, isotopic derivatives, and stereoisomers thereof:

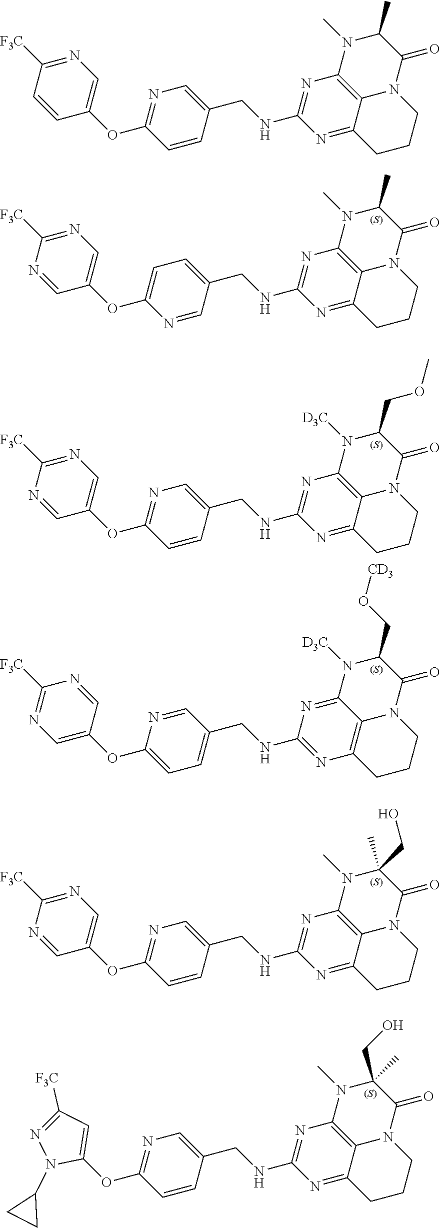

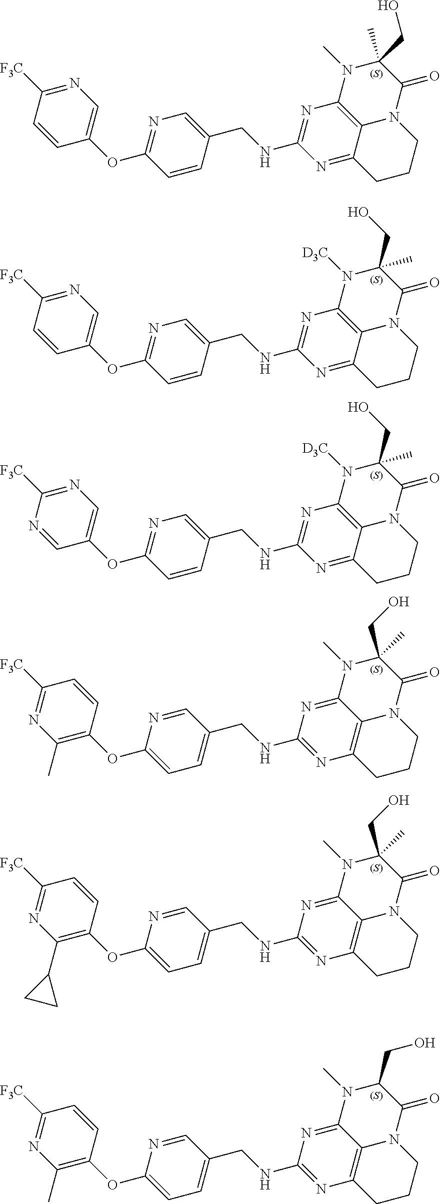

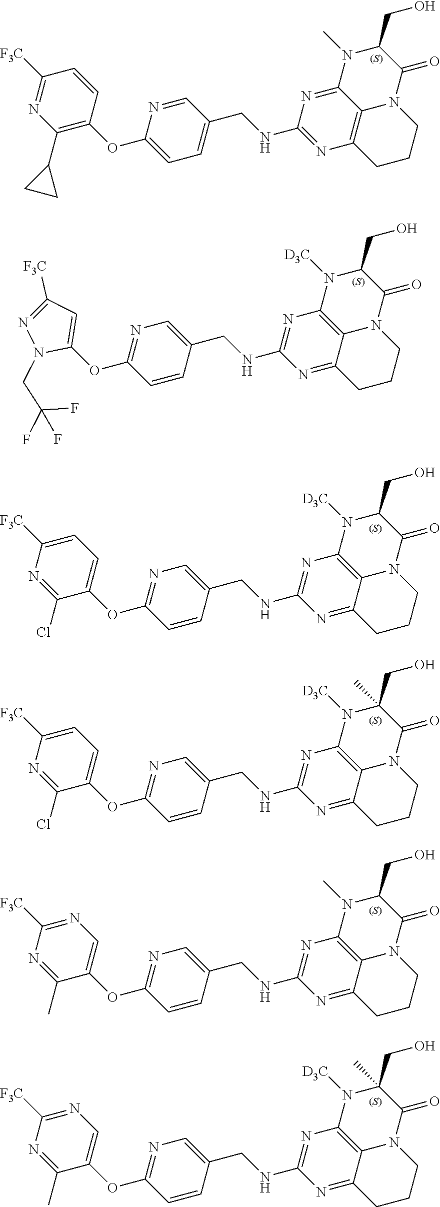

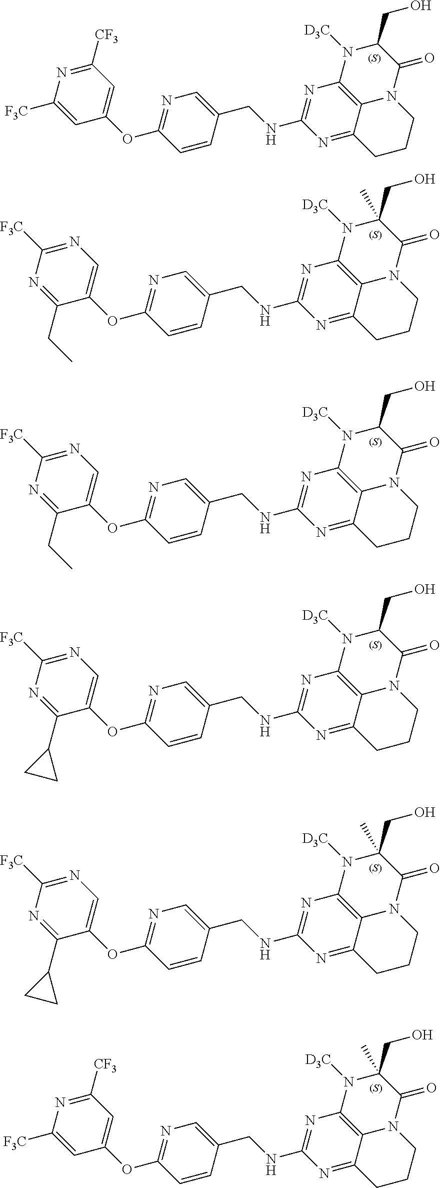

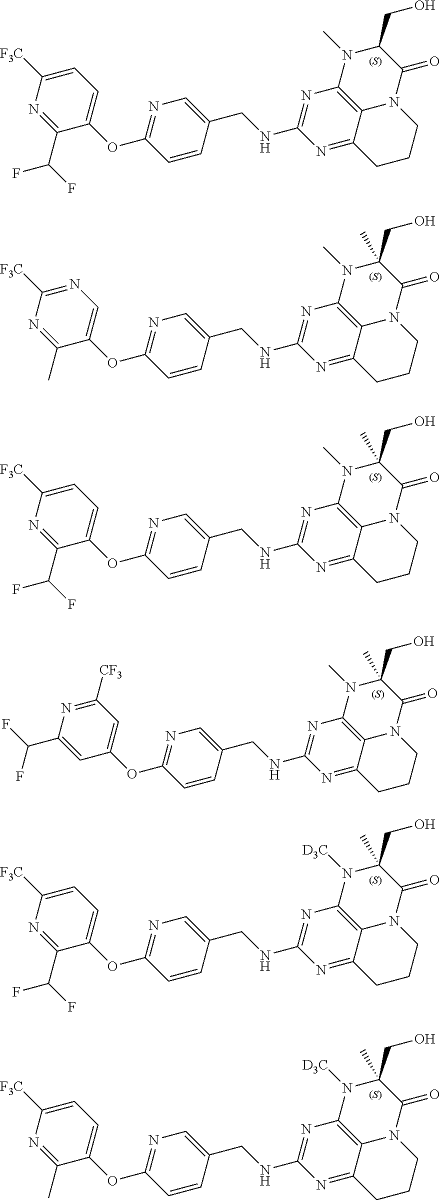

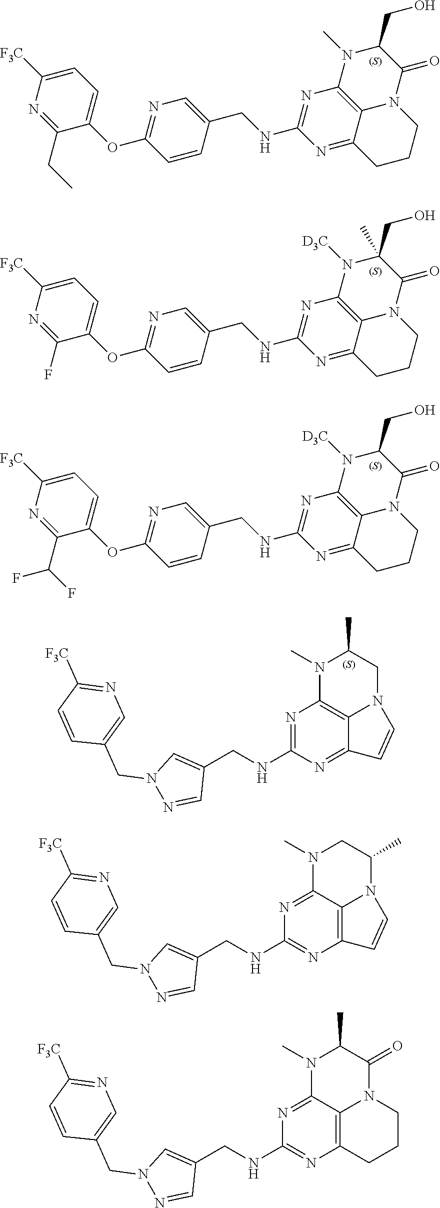

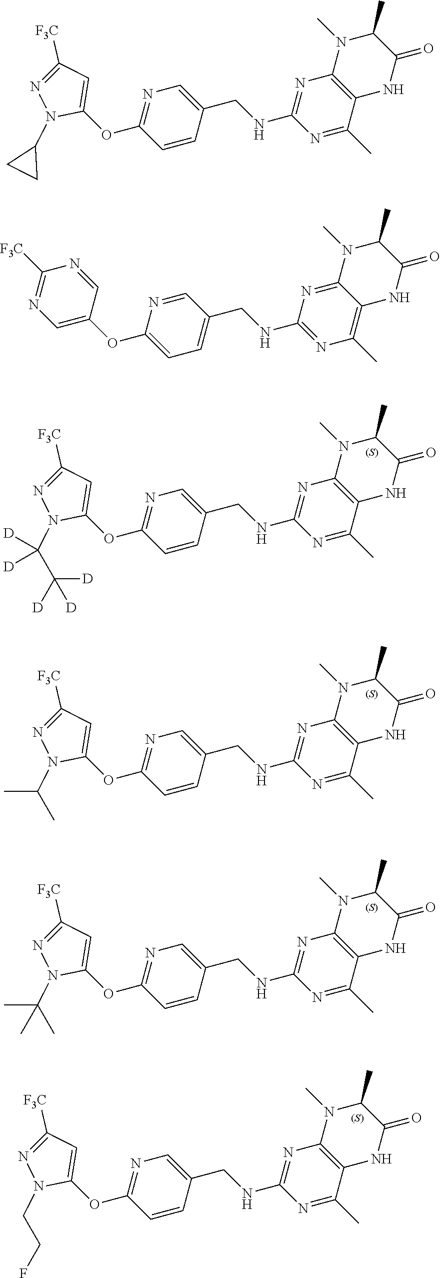

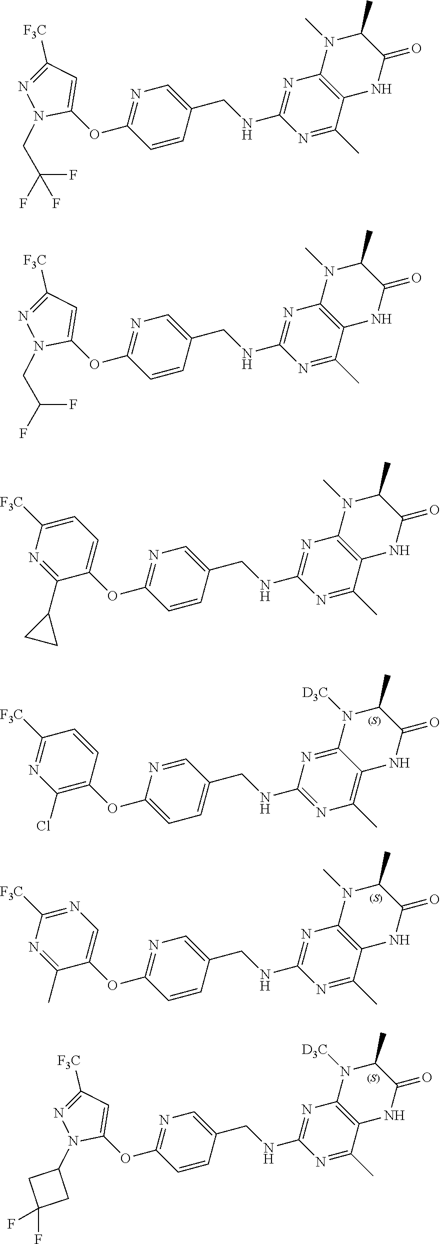

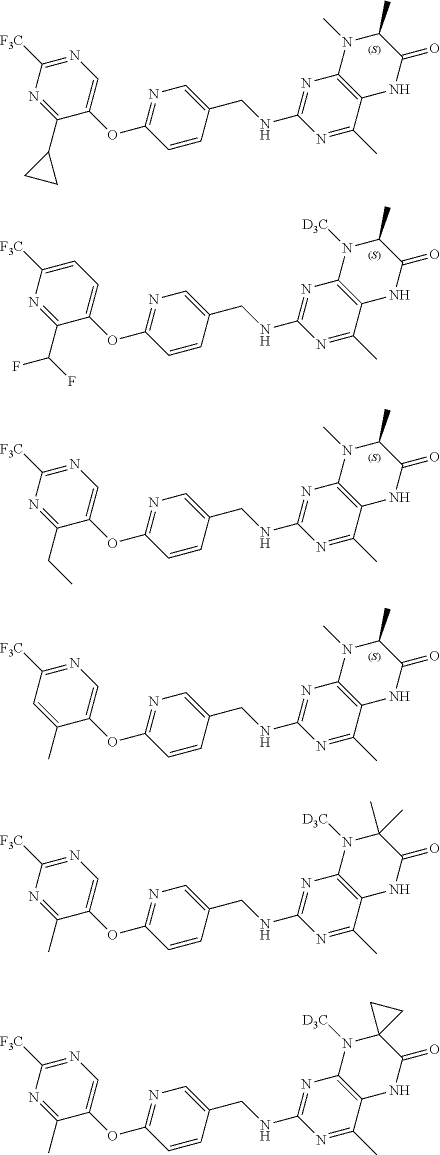

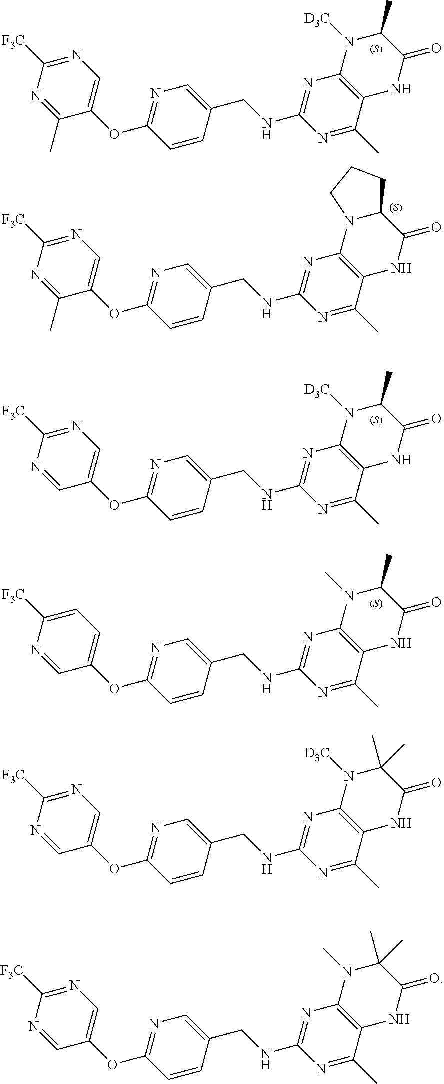

17. The method according to claim 2, wherein the Wnt pathway inhibitor is selected from the following compounds or pharmaceutically acceptable salts, isotopic derivatives, and stereoisomers thereof:

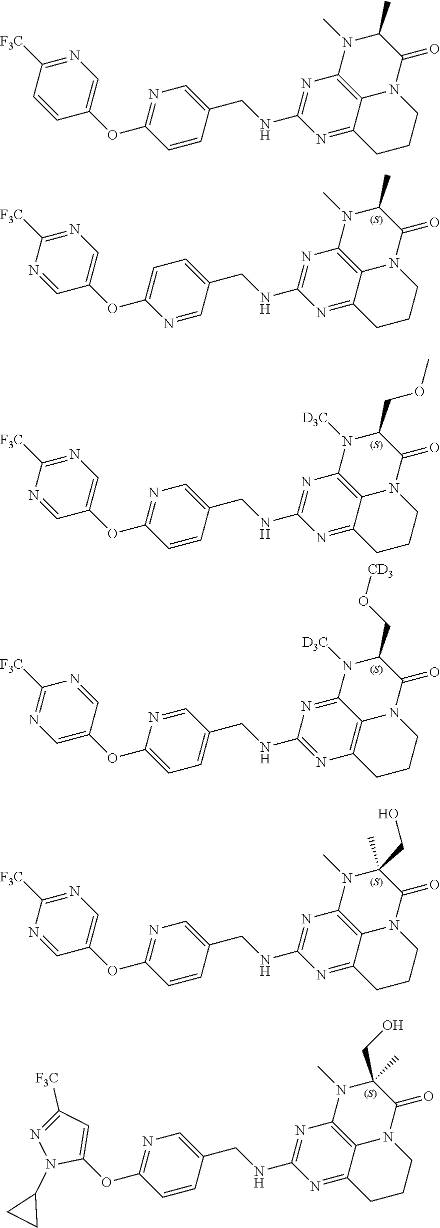

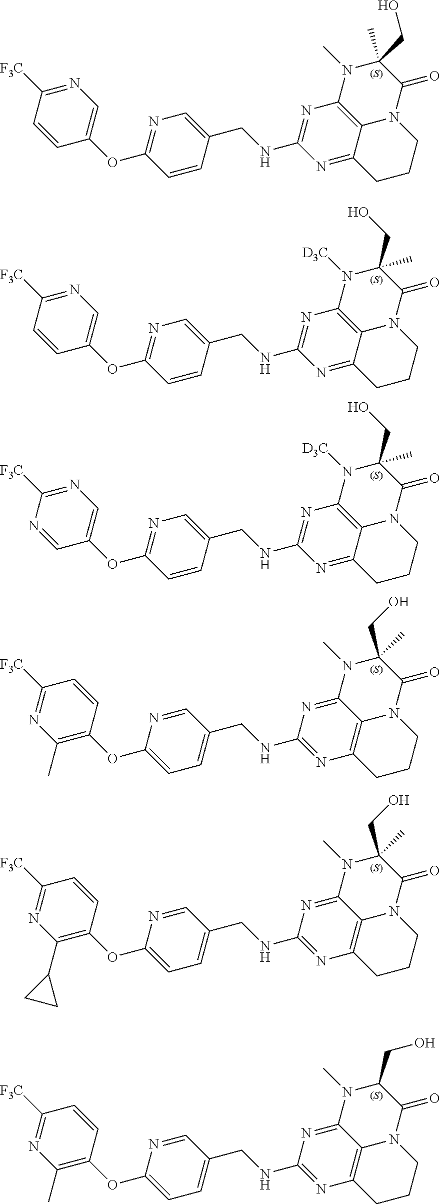

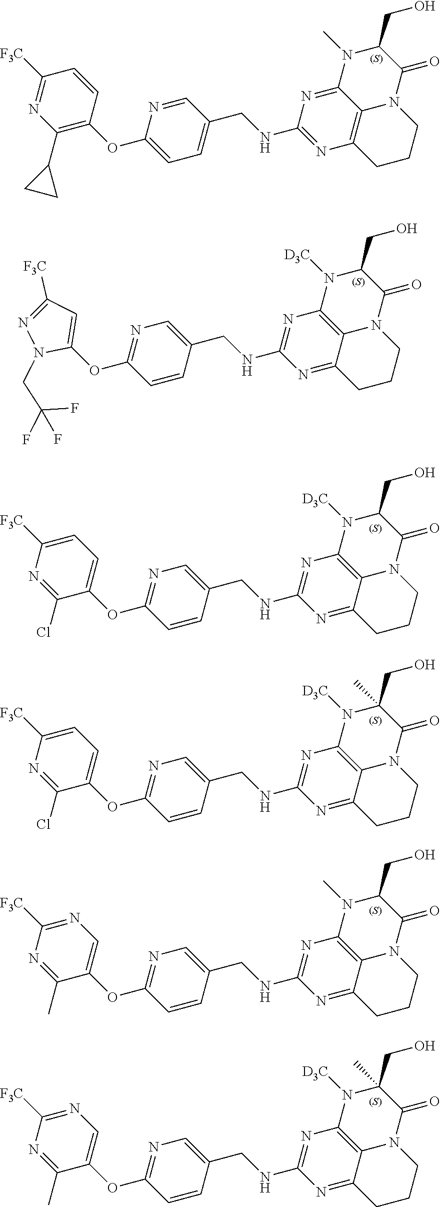

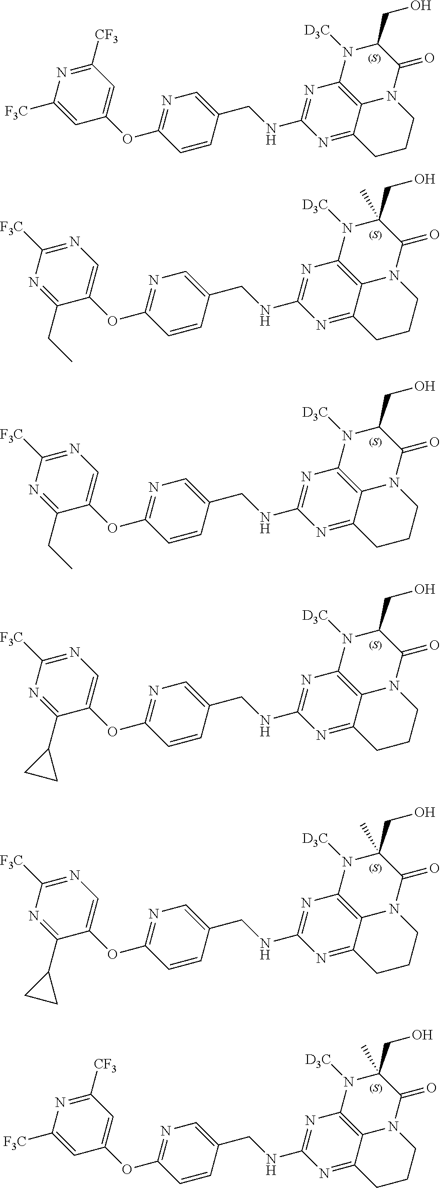

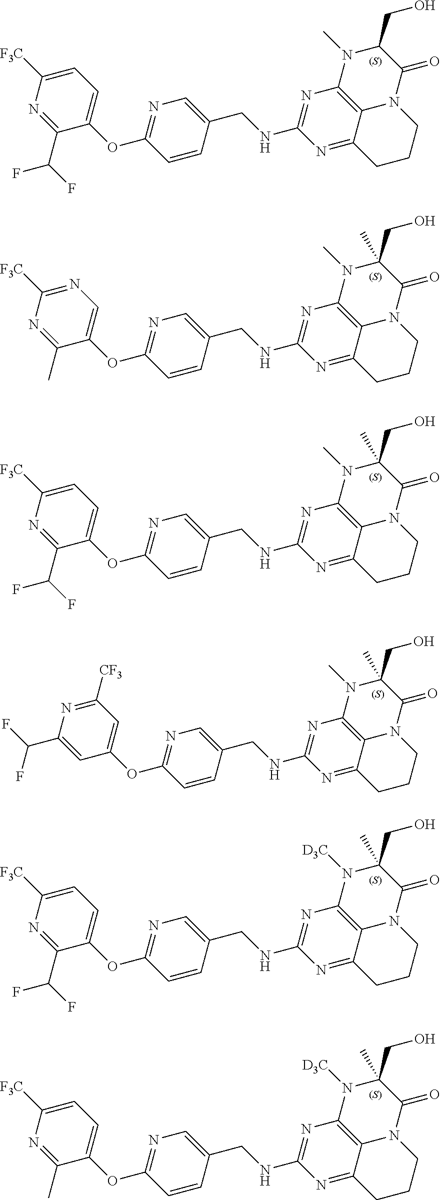

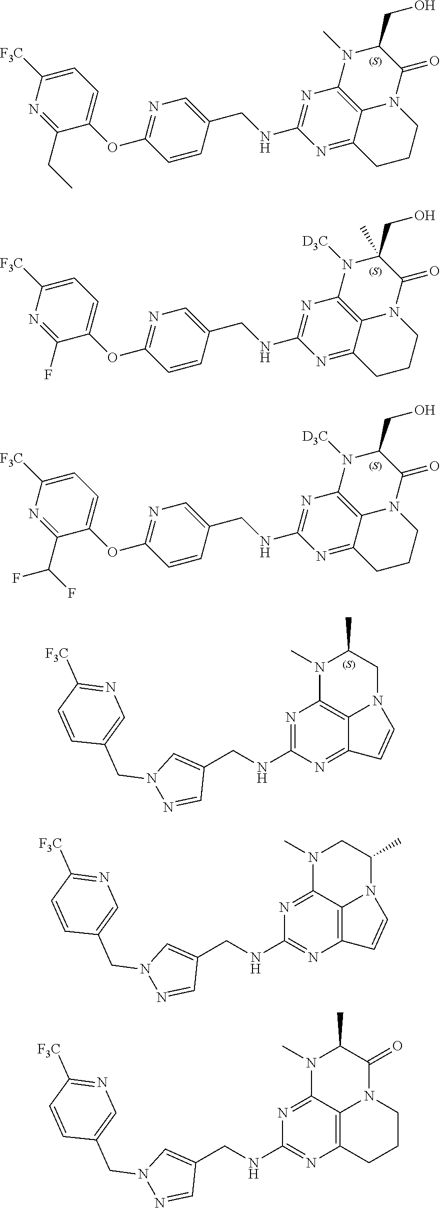

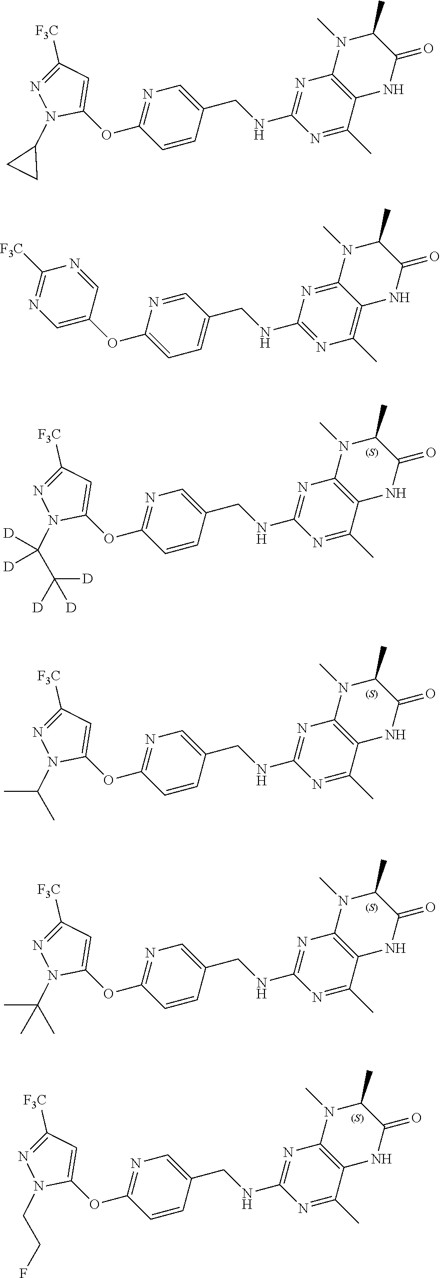

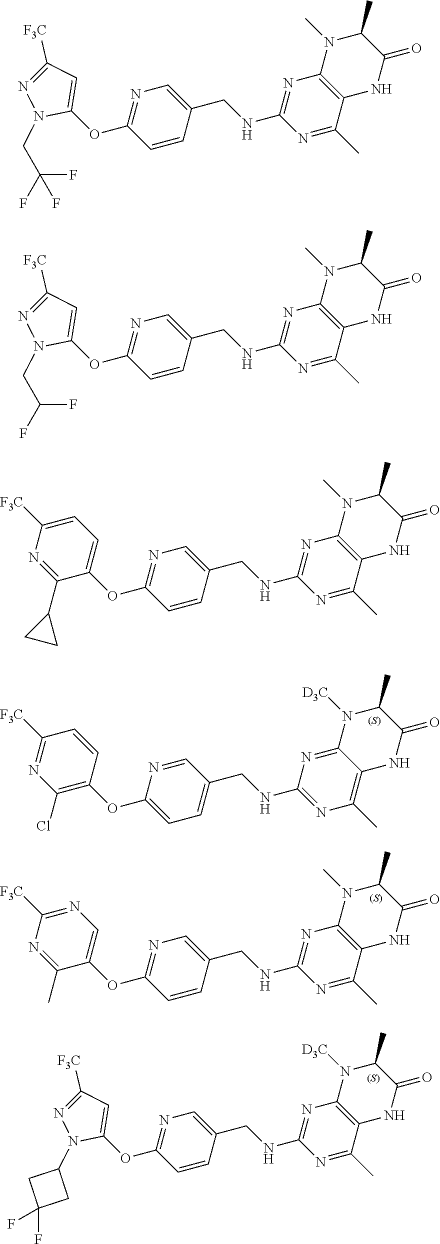

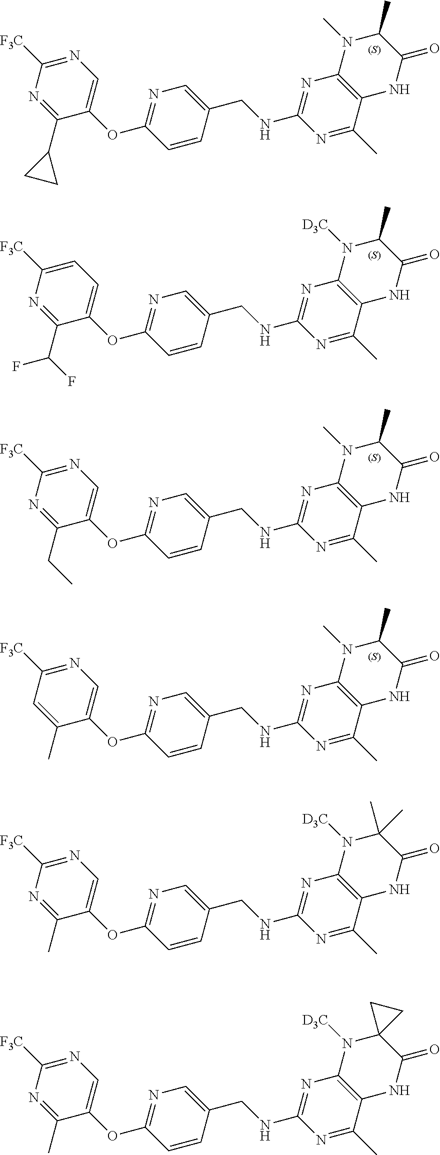

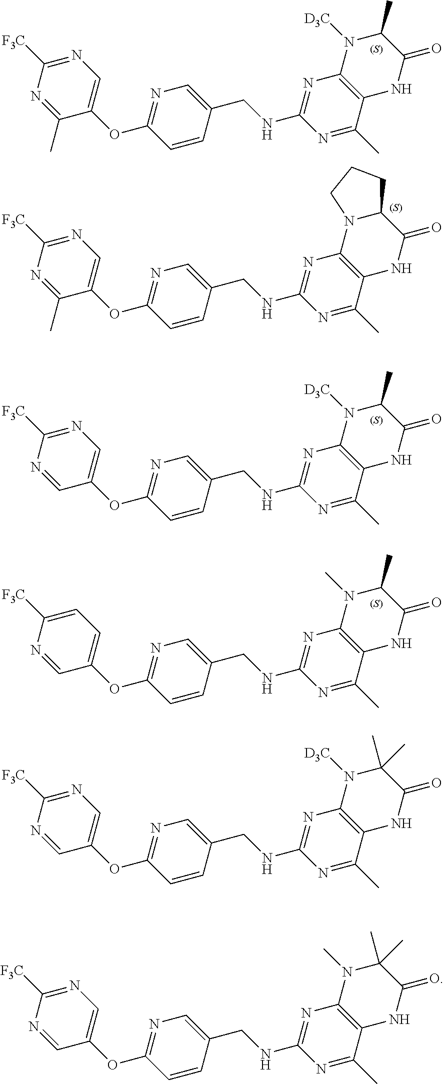

18. The method according to claim 3, wherein the Wnt pathway inhibitor is selected from the following compounds or pharmaceutically acceptable salts, isotopic derivatives, and stereoisomers thereof:

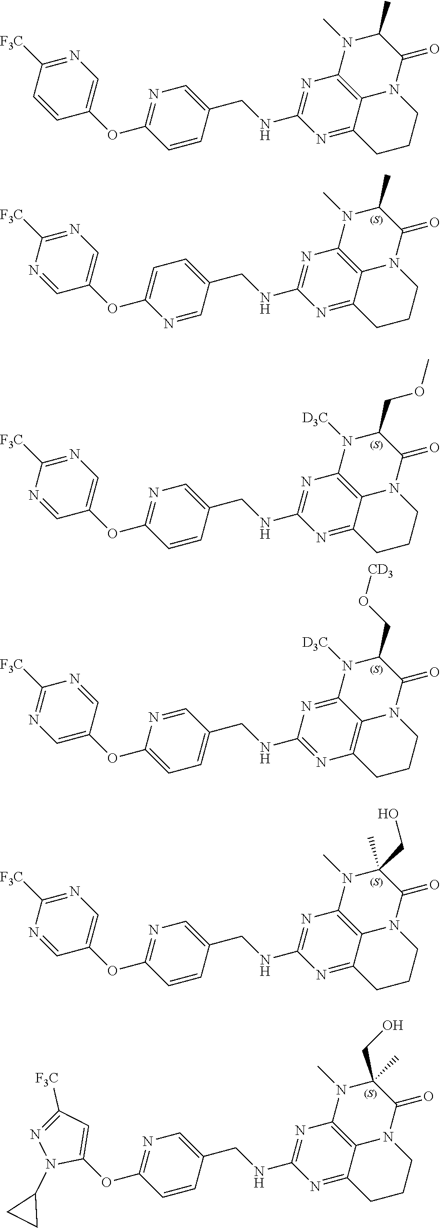

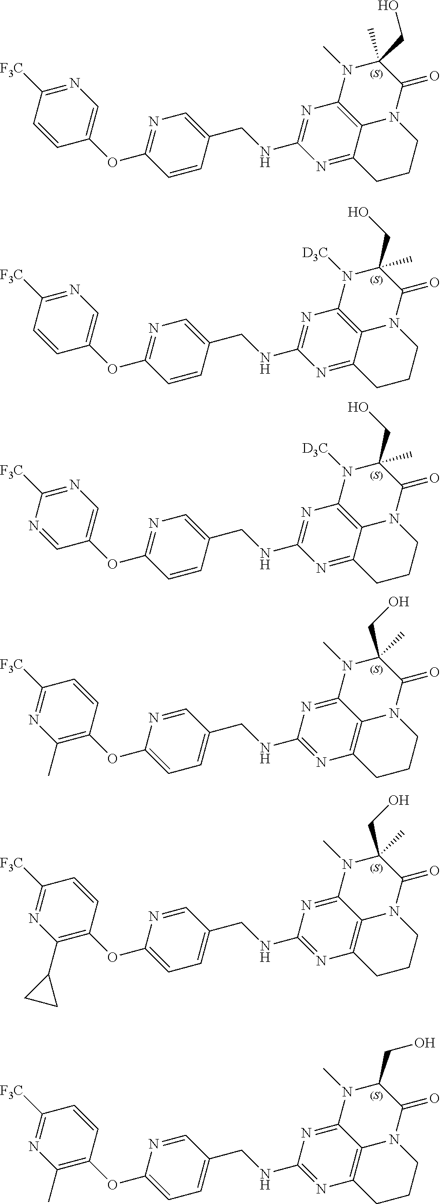

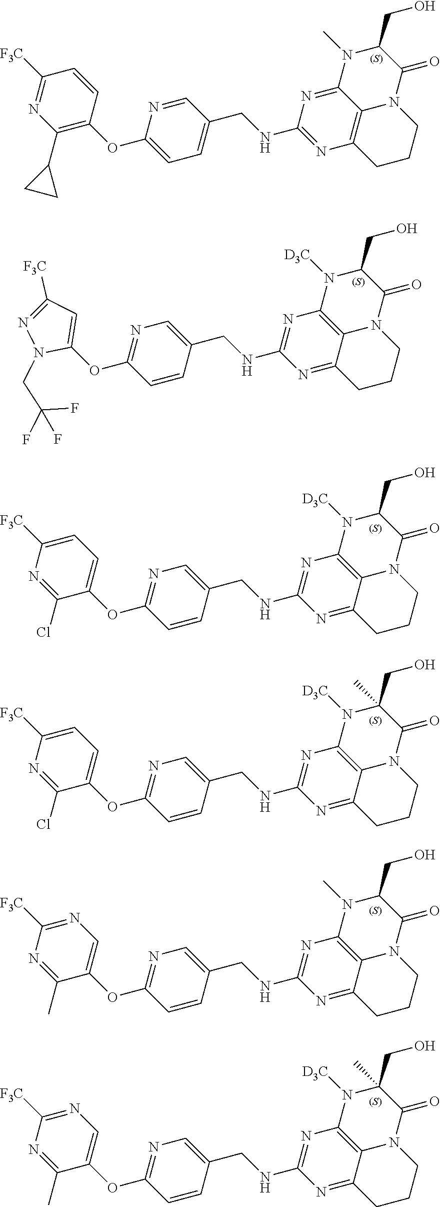

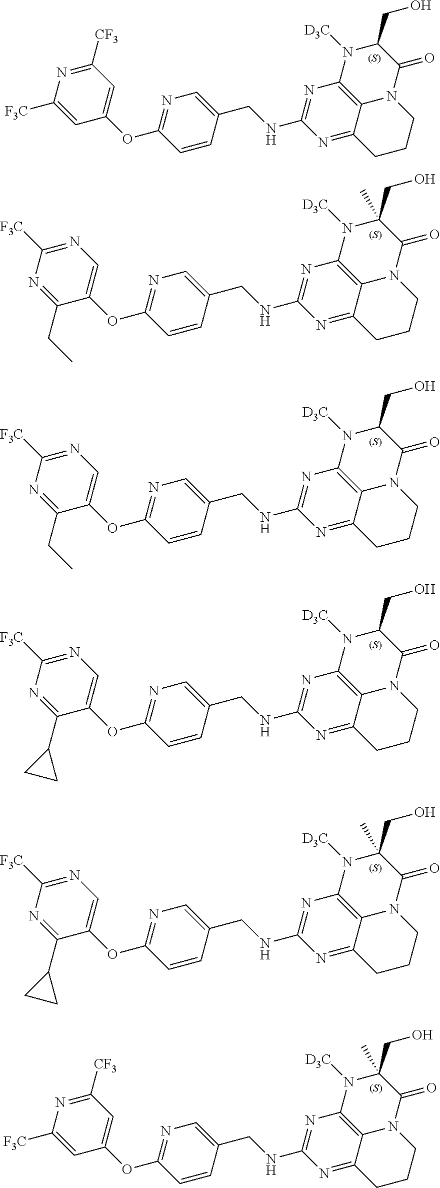

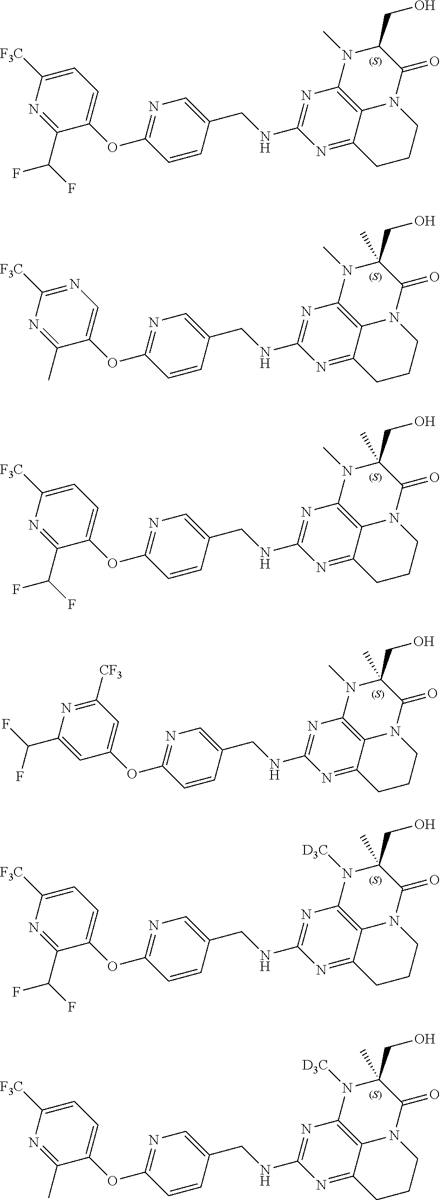

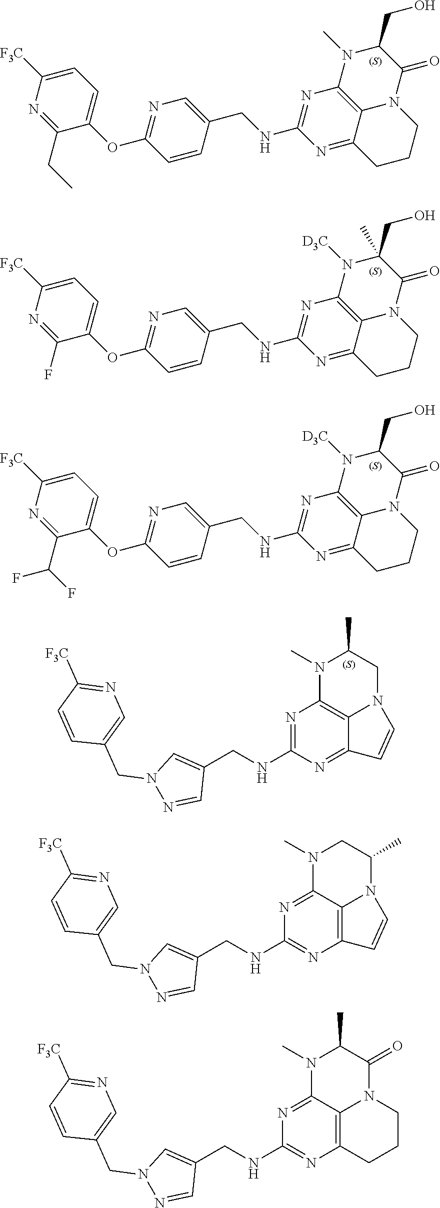

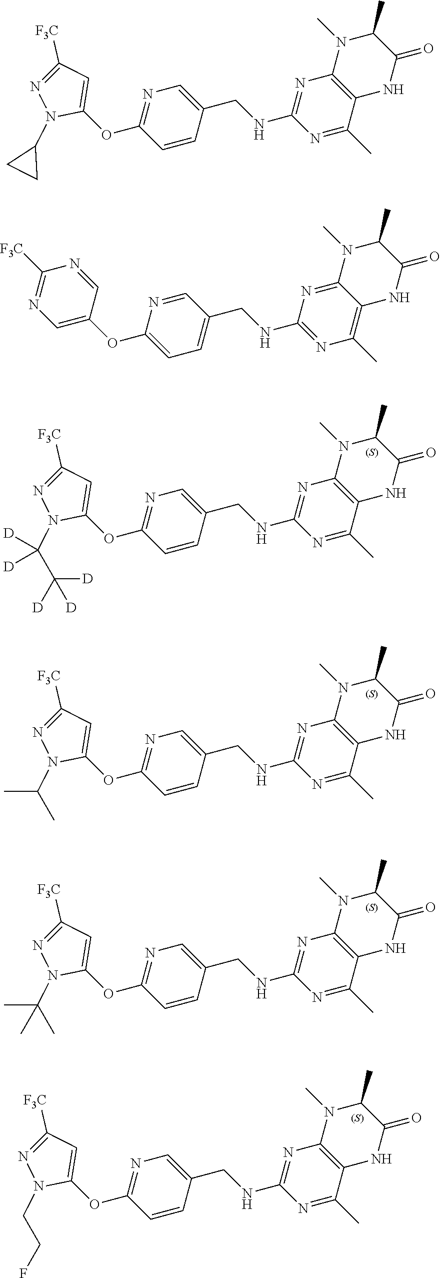

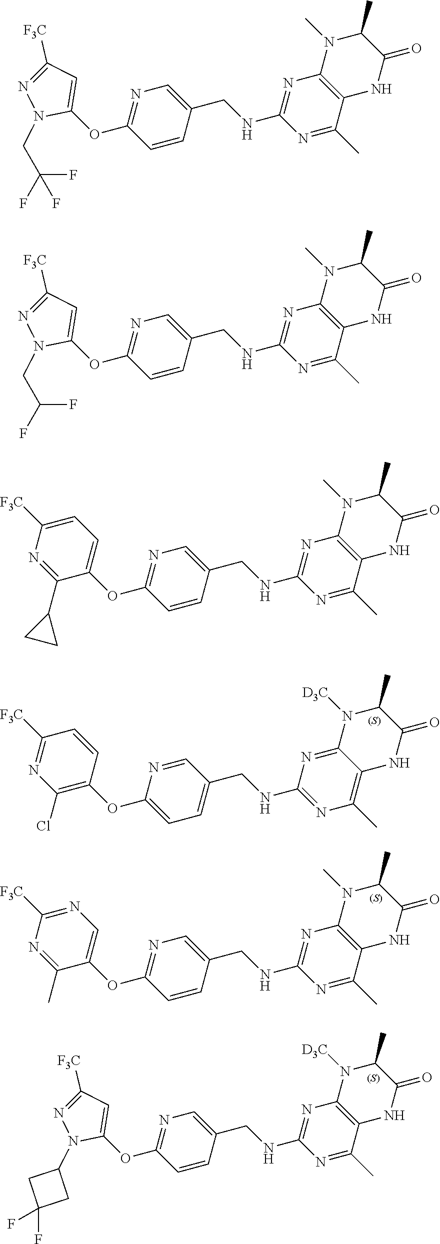

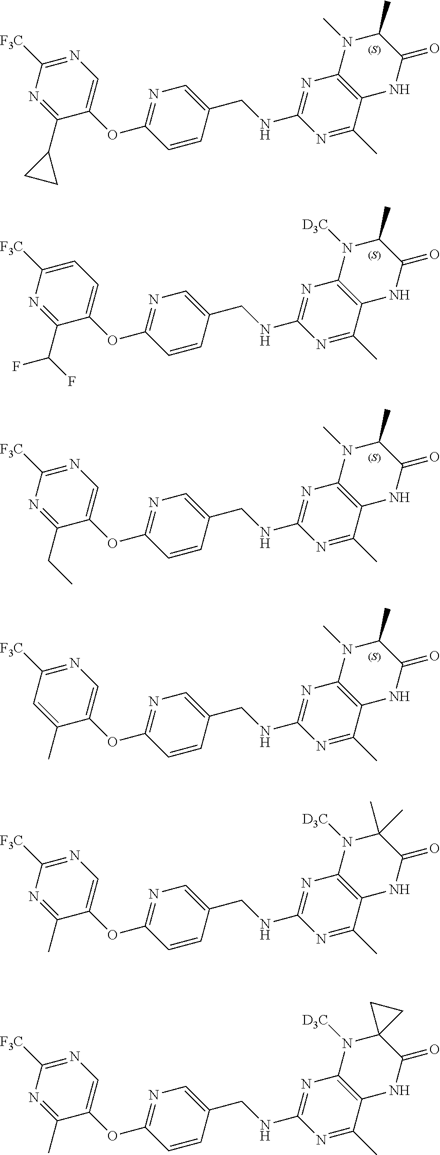

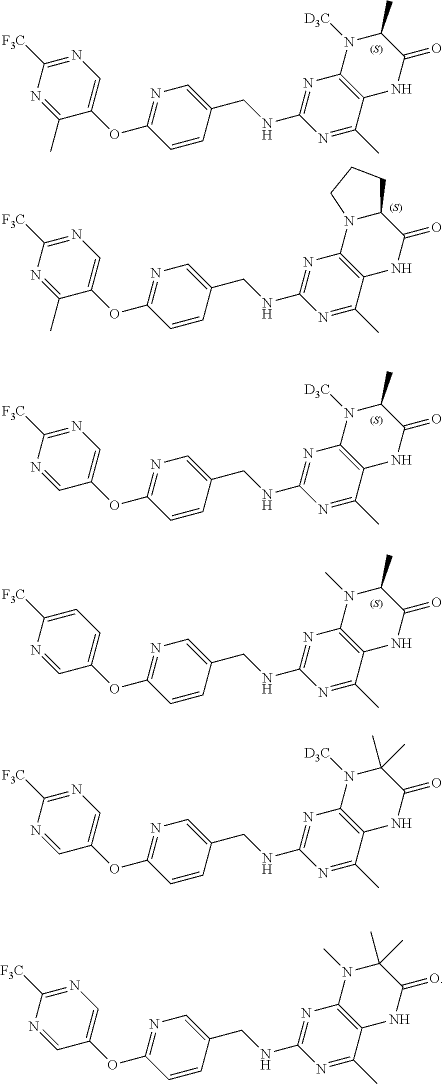

19. The method according to claim 4, wherein the Wnt pathway inhibitor is selected from the following compounds or pharmaceutically acceptable salts, isotopic derivatives, and stereoisomers thereof:

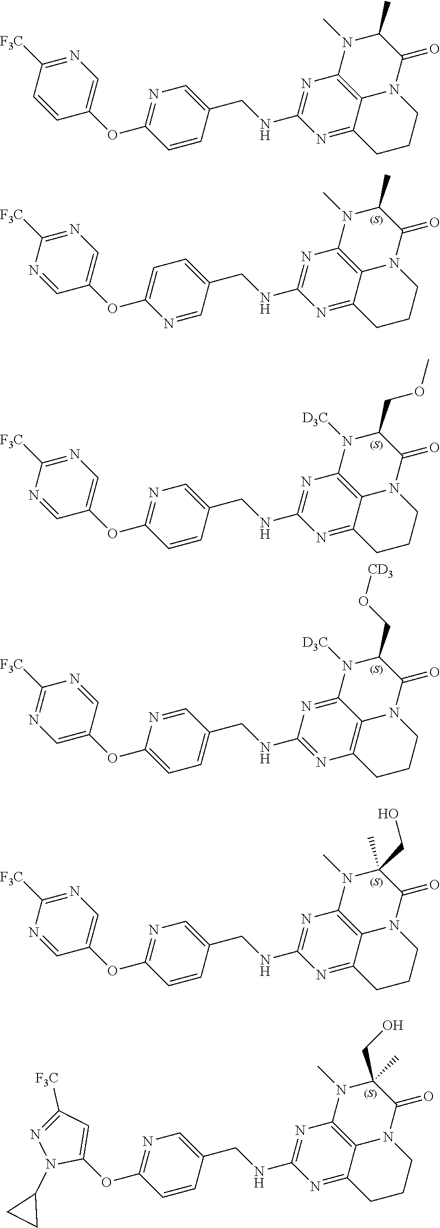

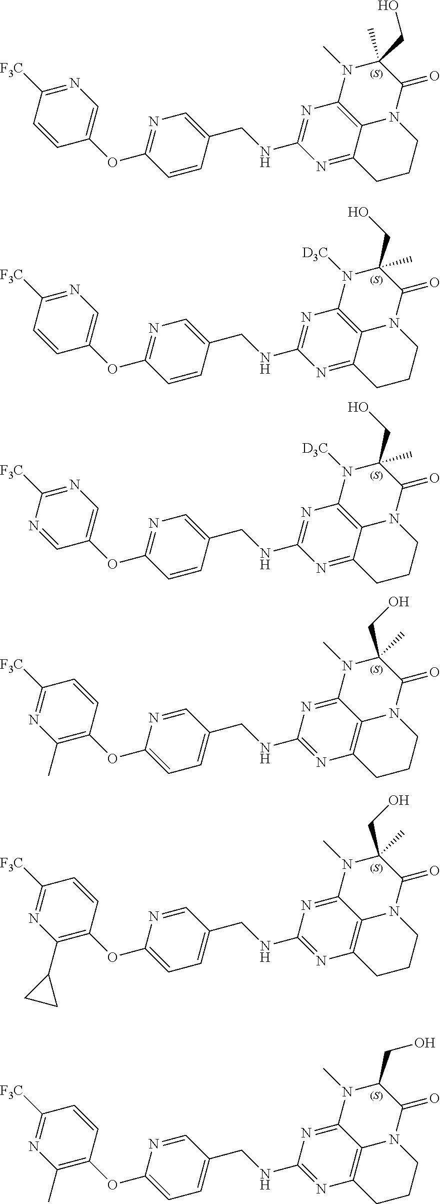

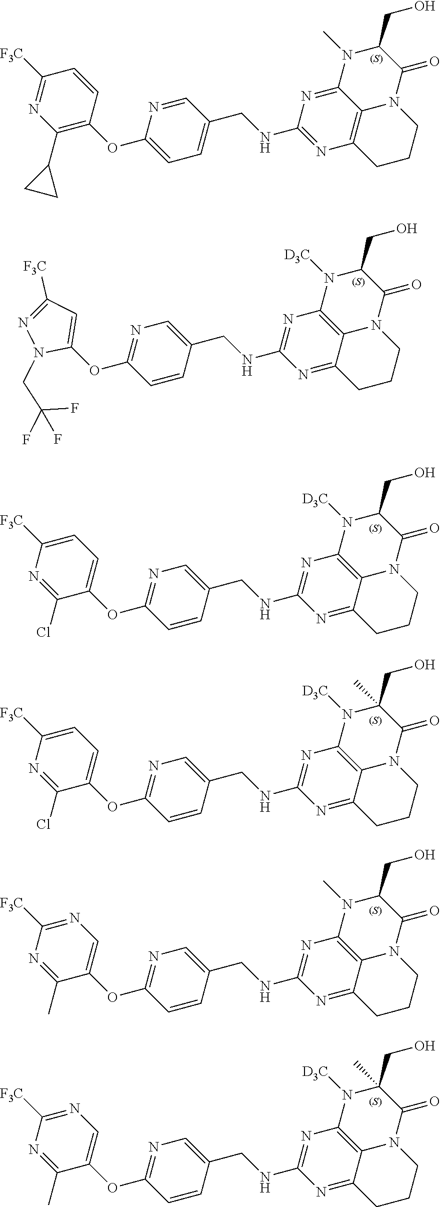

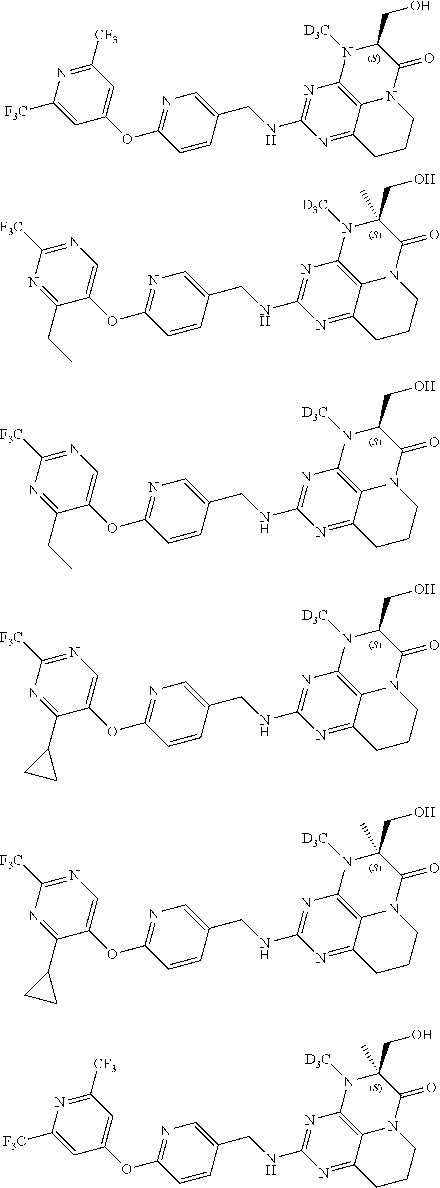

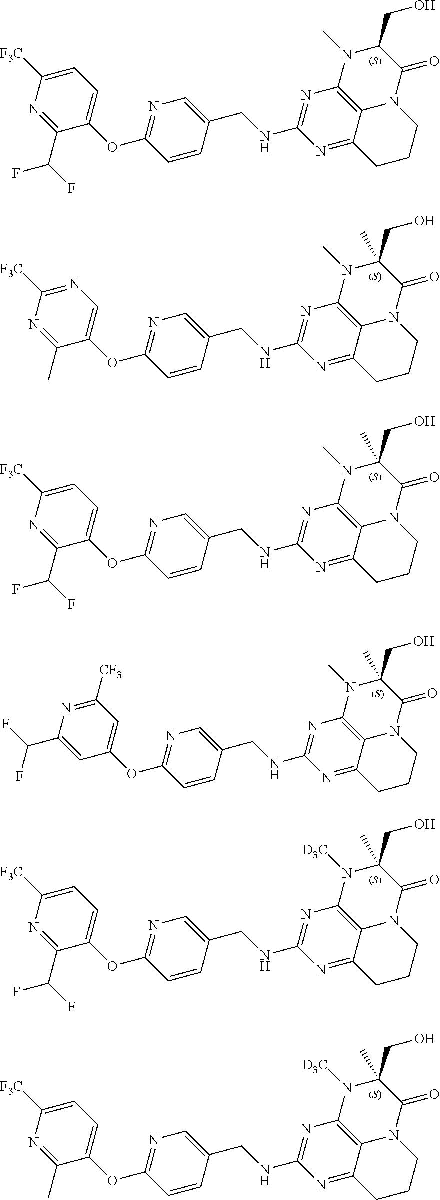

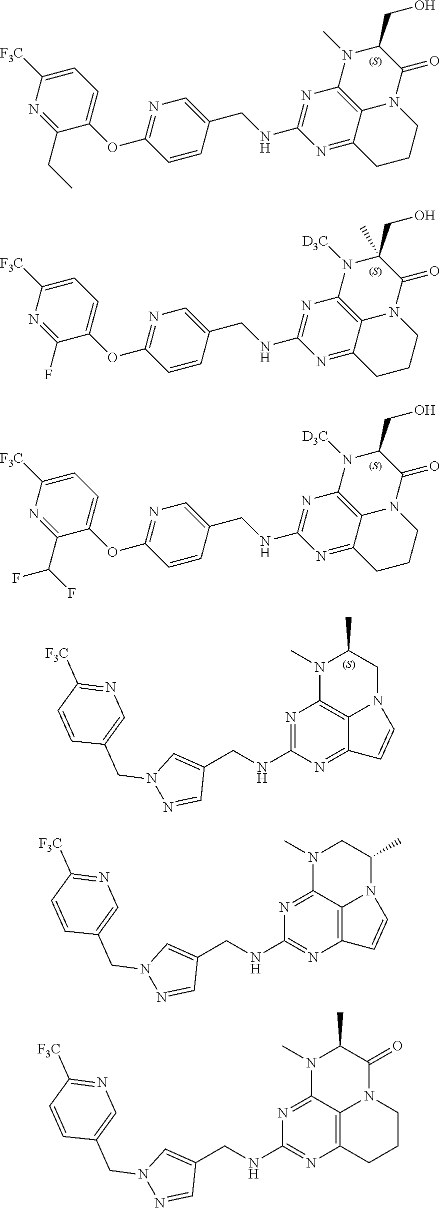

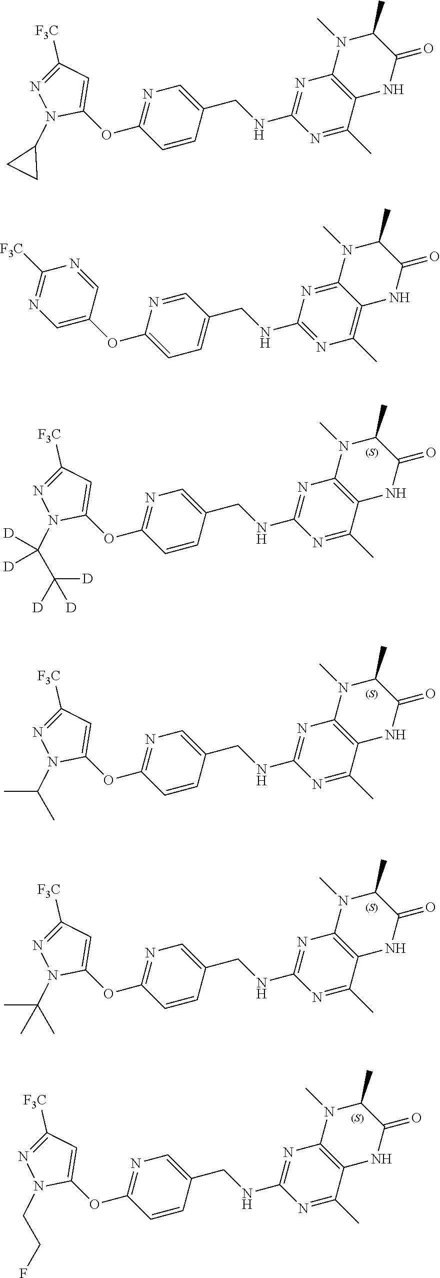

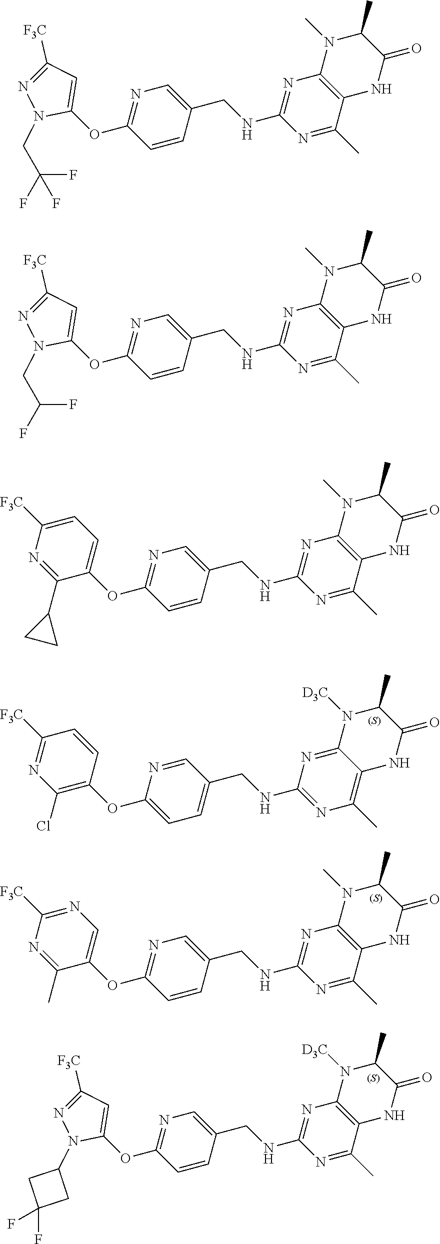

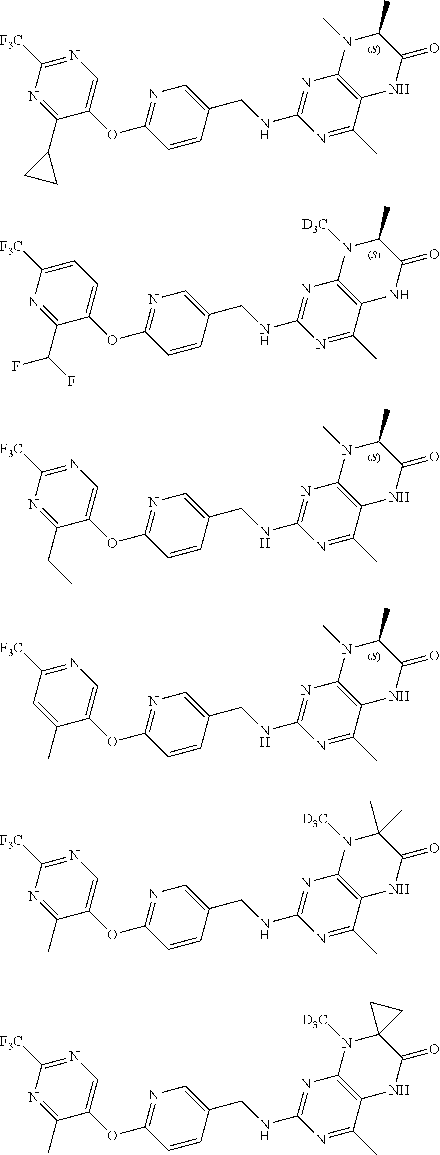

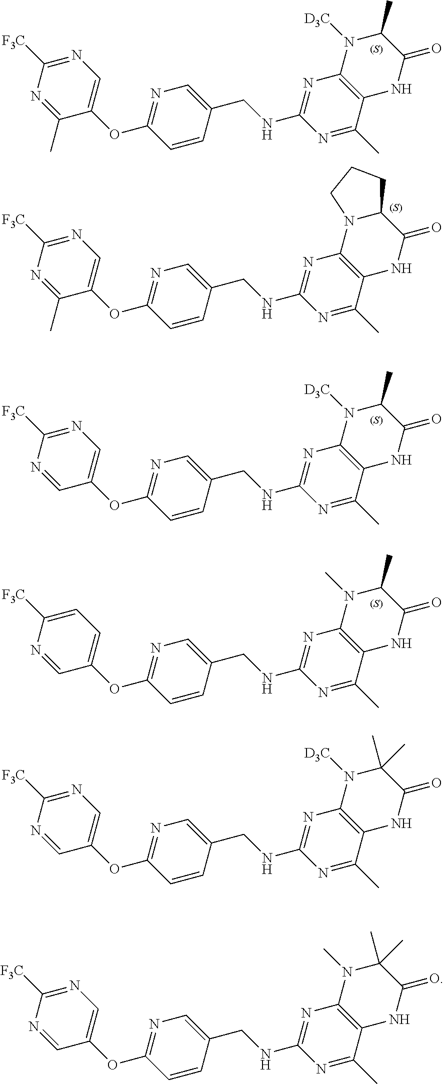

20. The method according to claim 5, wherein the Wnt pathway inhibitor is selected from the following compounds or pharmaceutically acceptable salts, isotopic derivatives, and stereoisomers thereof:

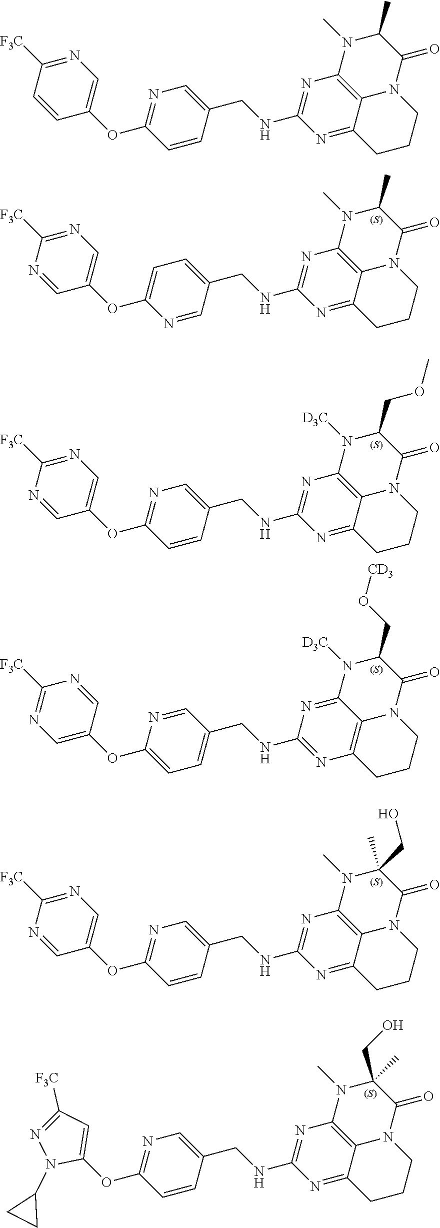

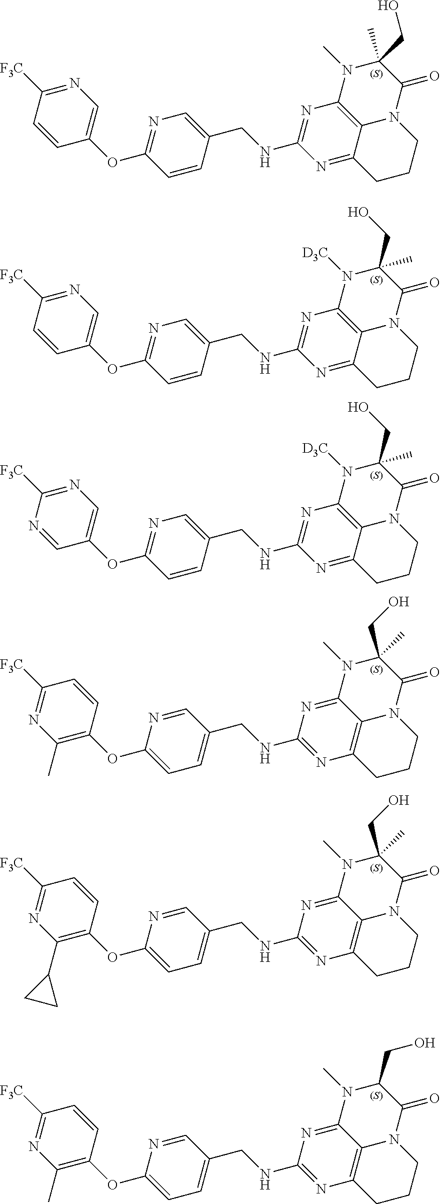

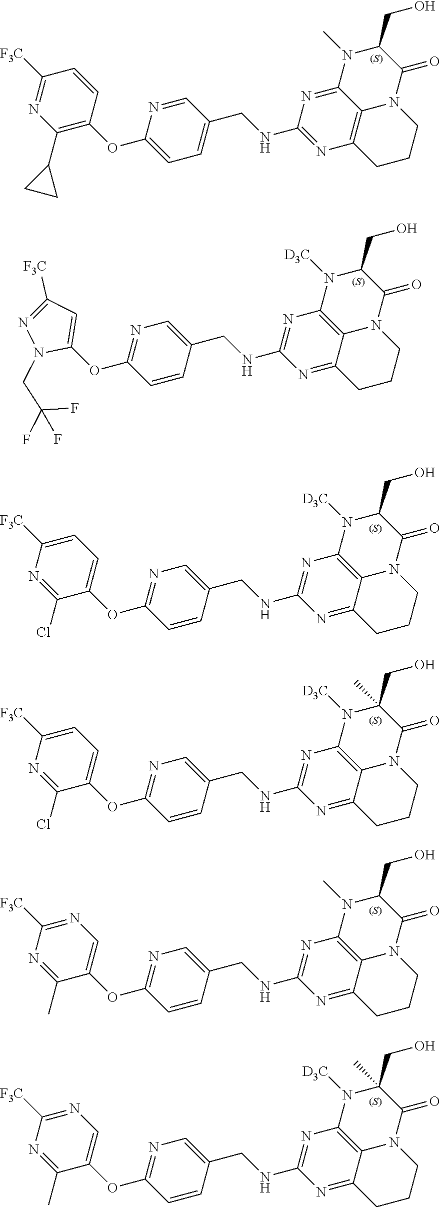

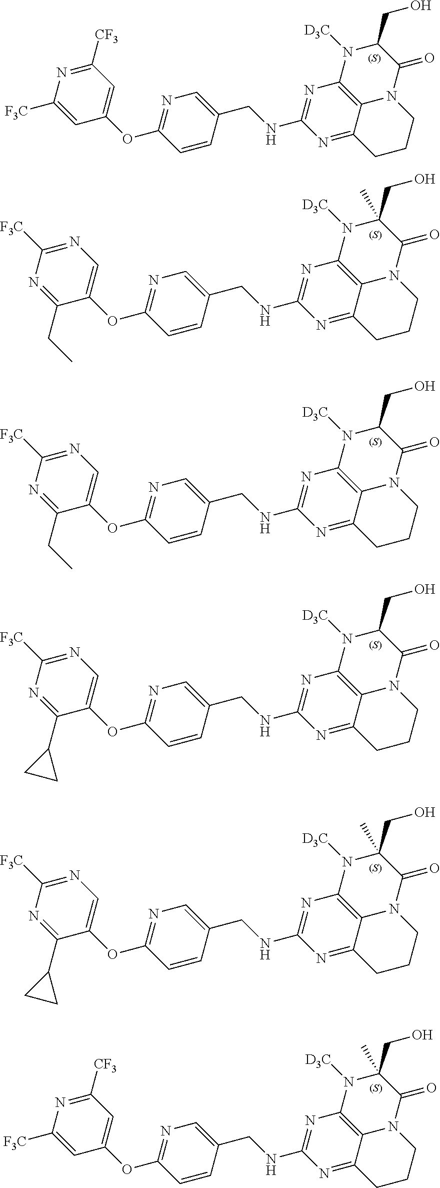

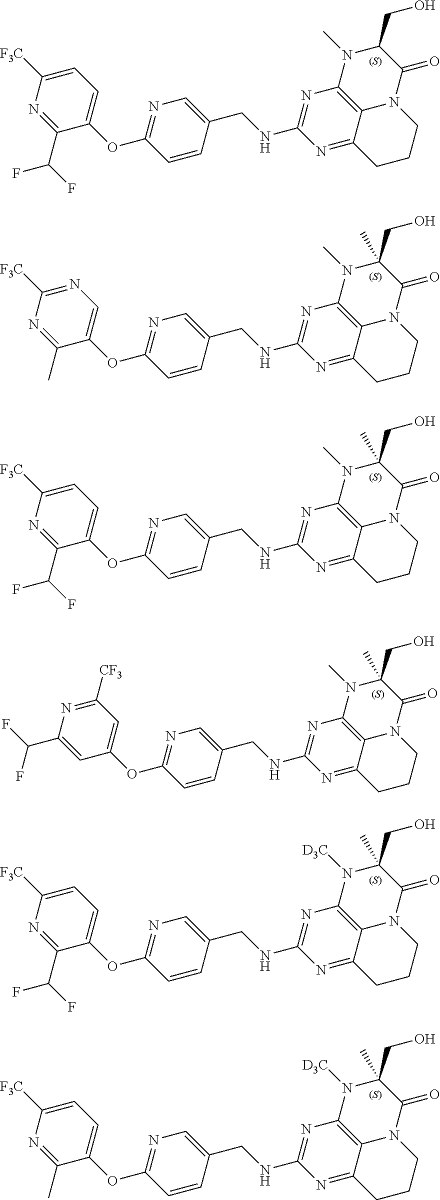

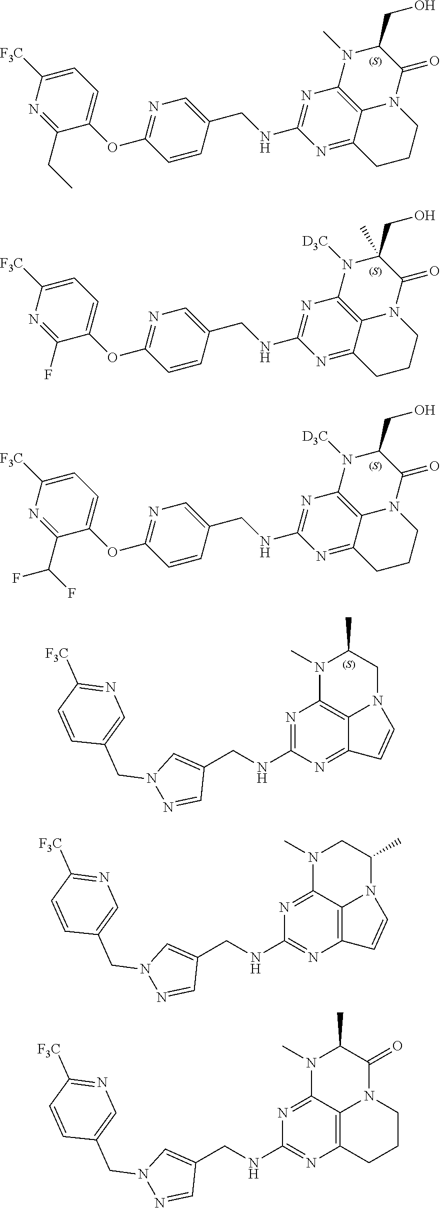

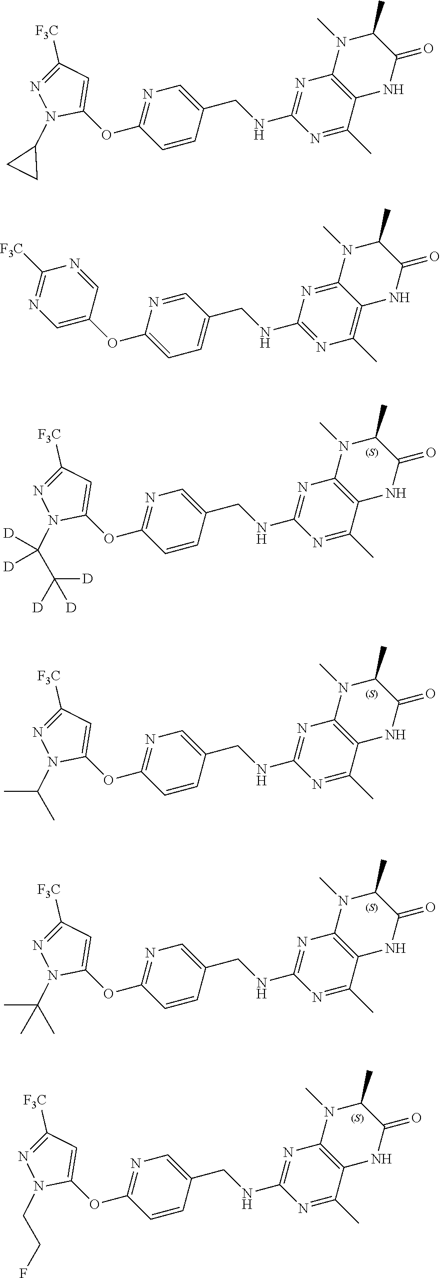

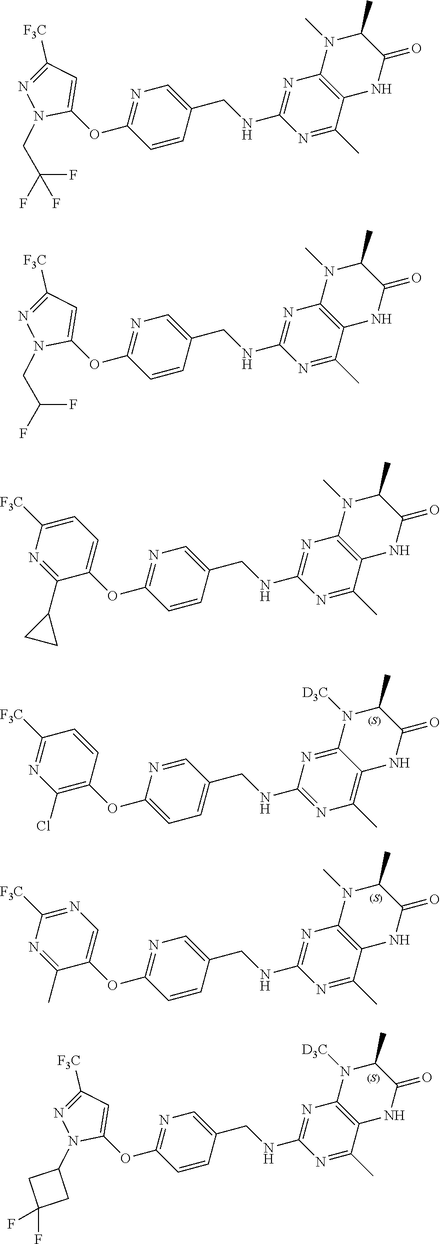

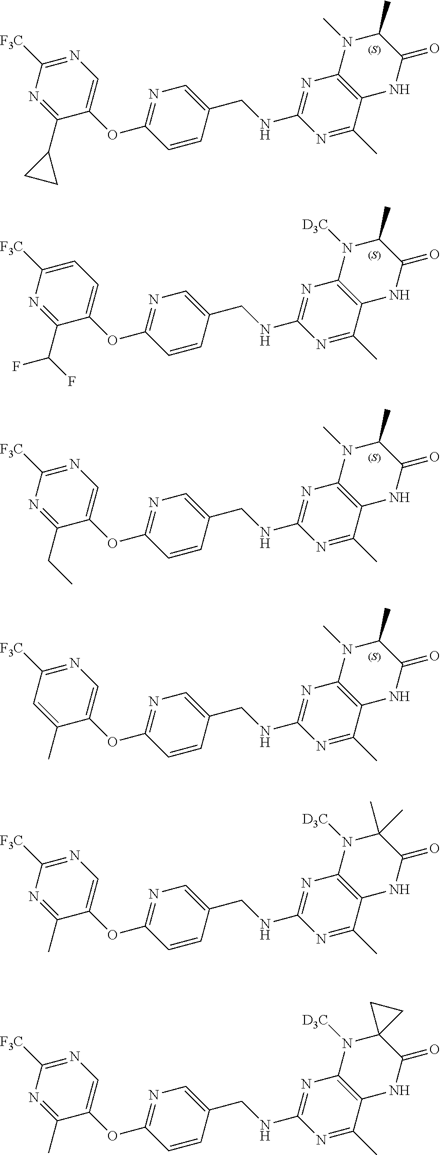

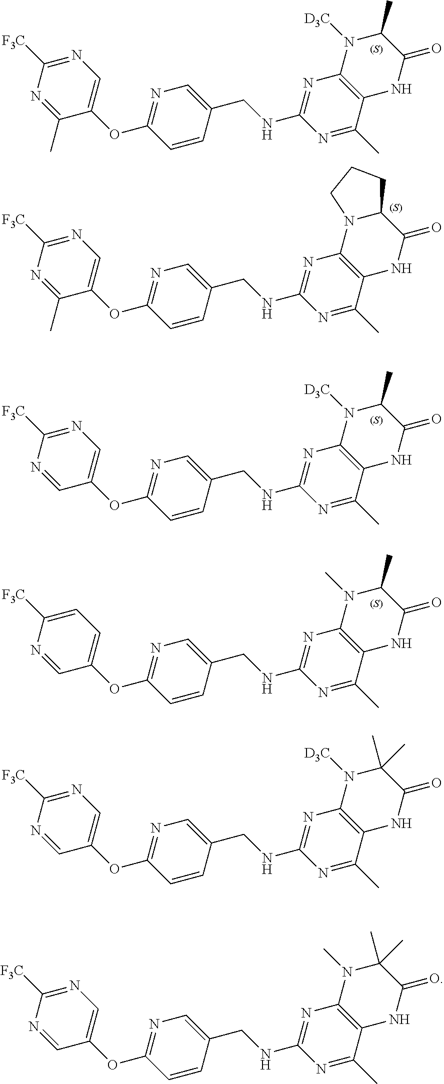

21. The method according to claim 6, wherein the Wnt pathway inhibitor is selected from the following compounds or pharmaceutically acceptable salts, isotopic derivatives, and stereoisomers thereof: 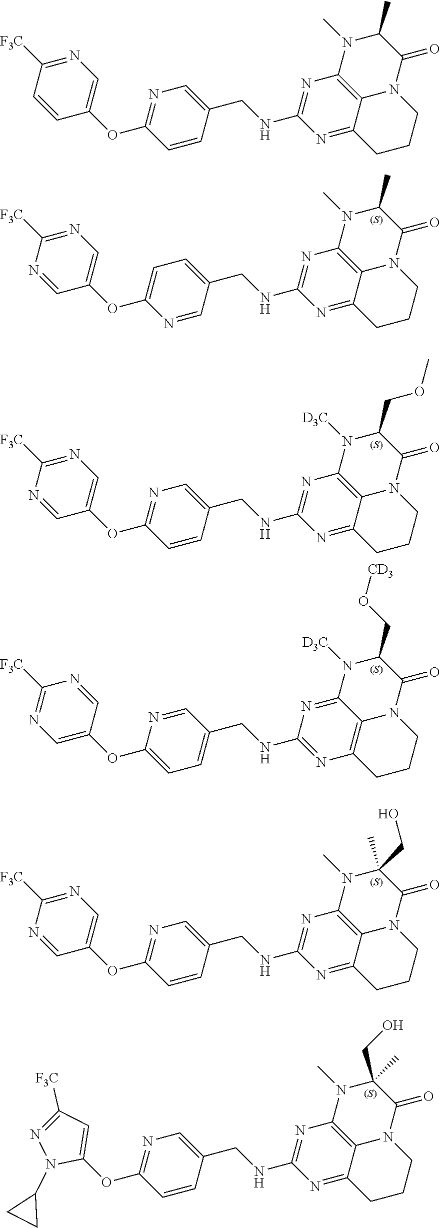 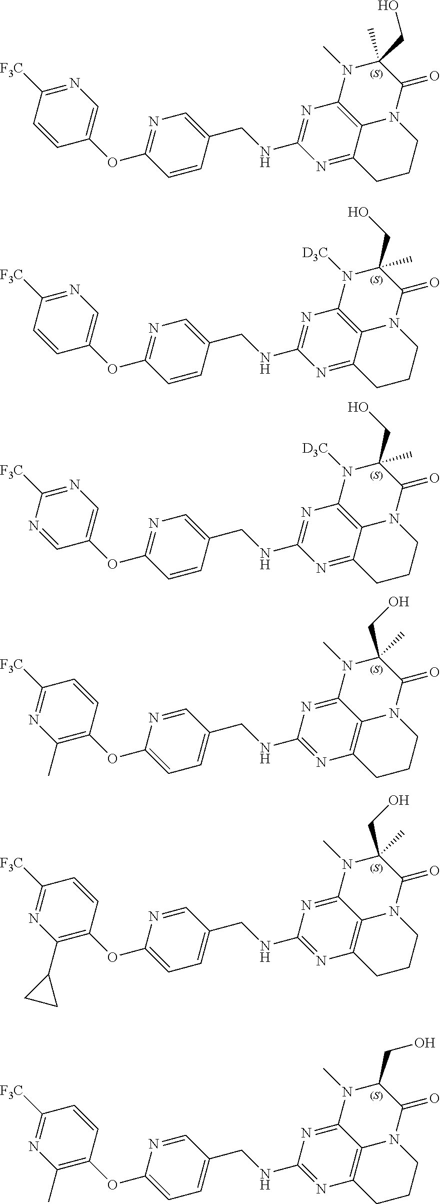 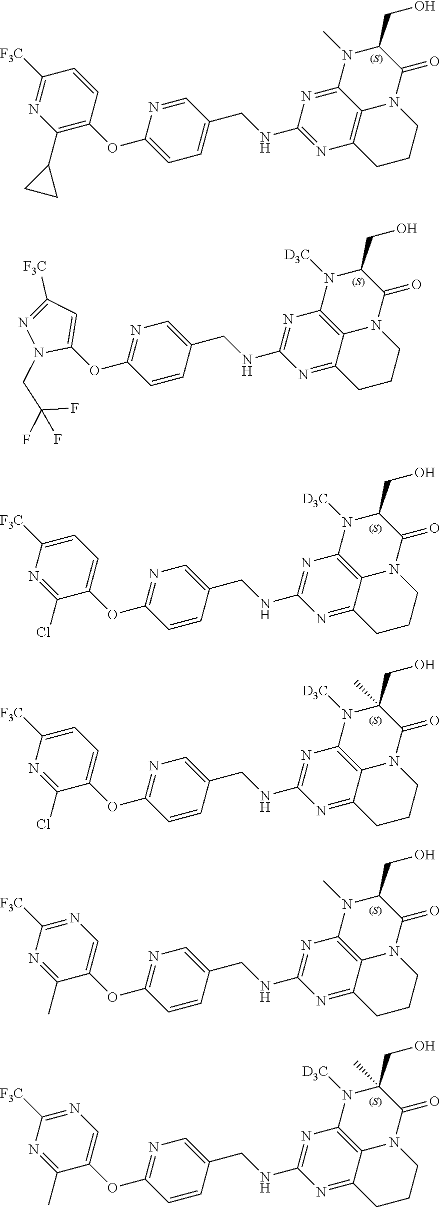 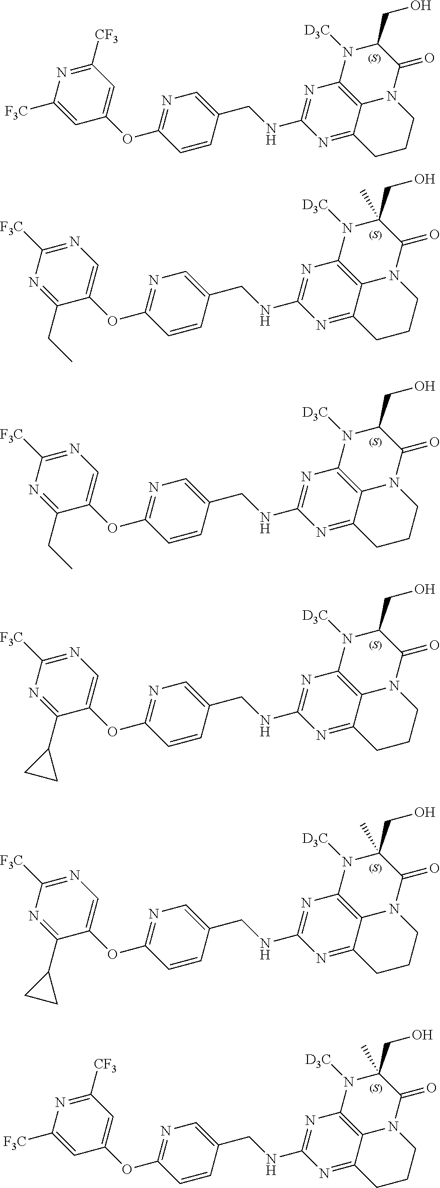 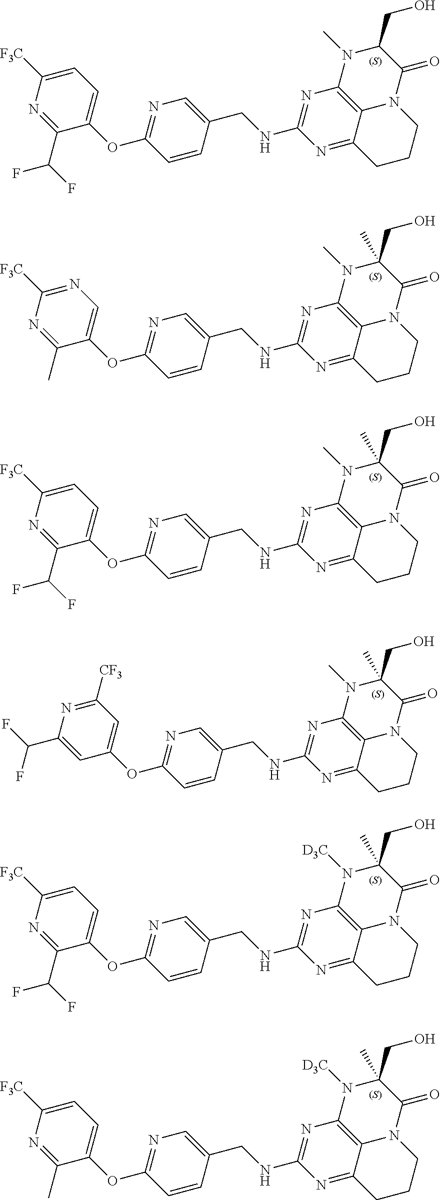 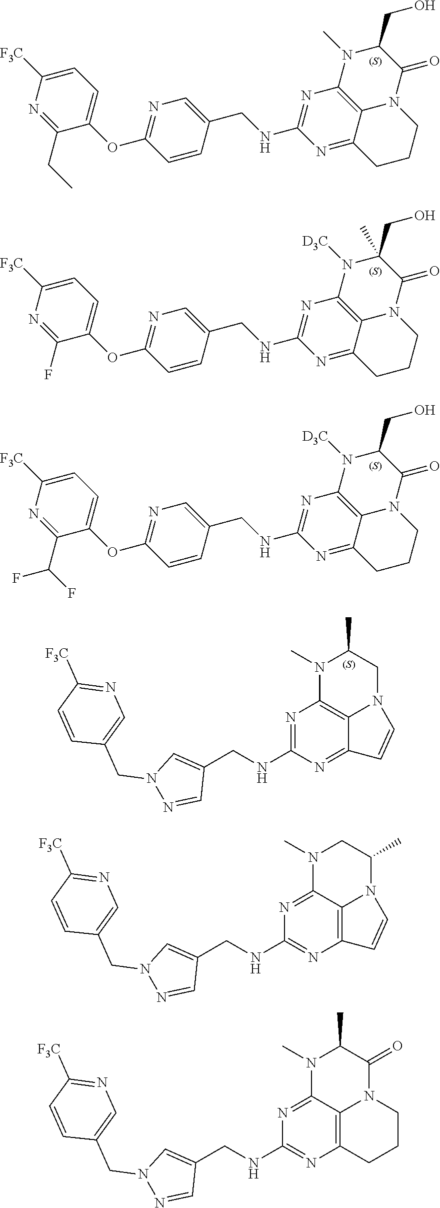 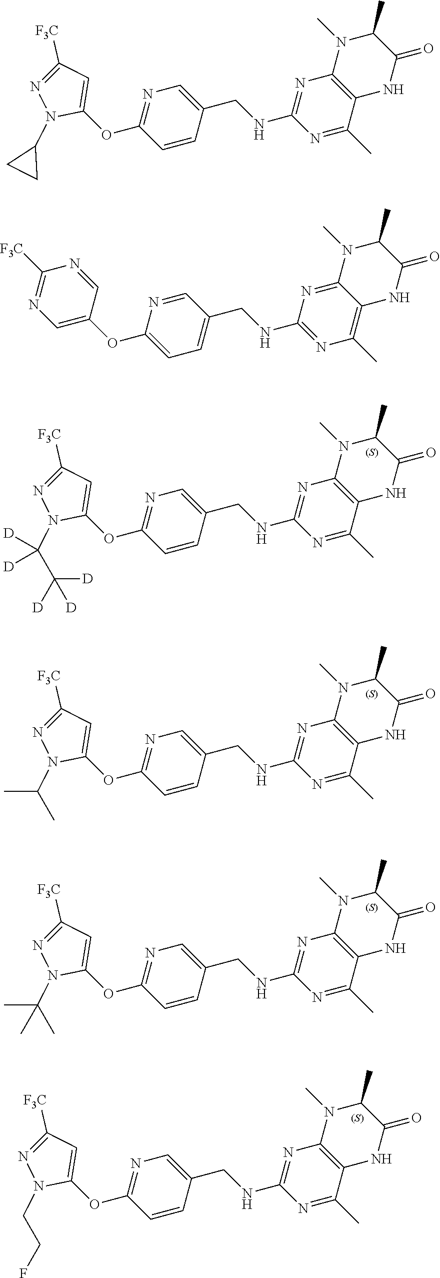 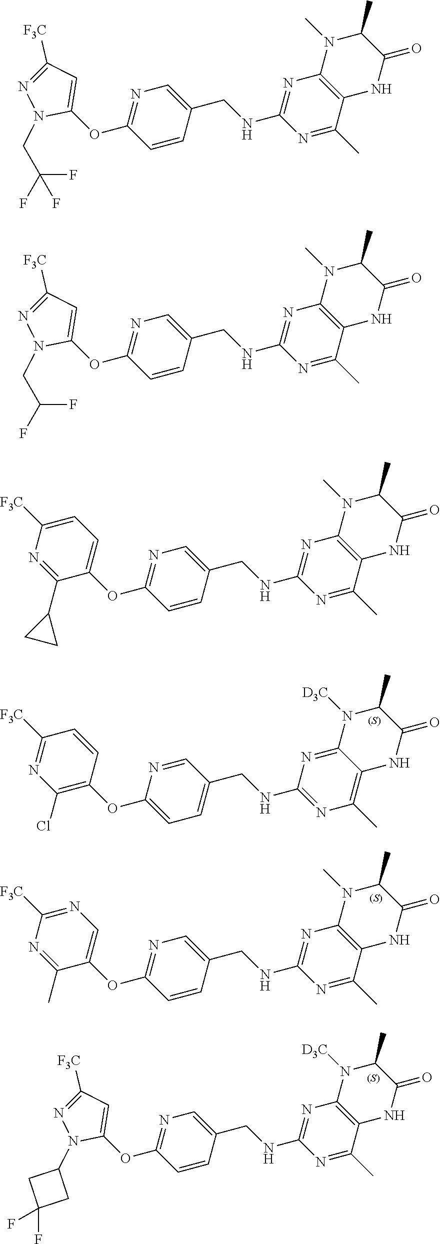 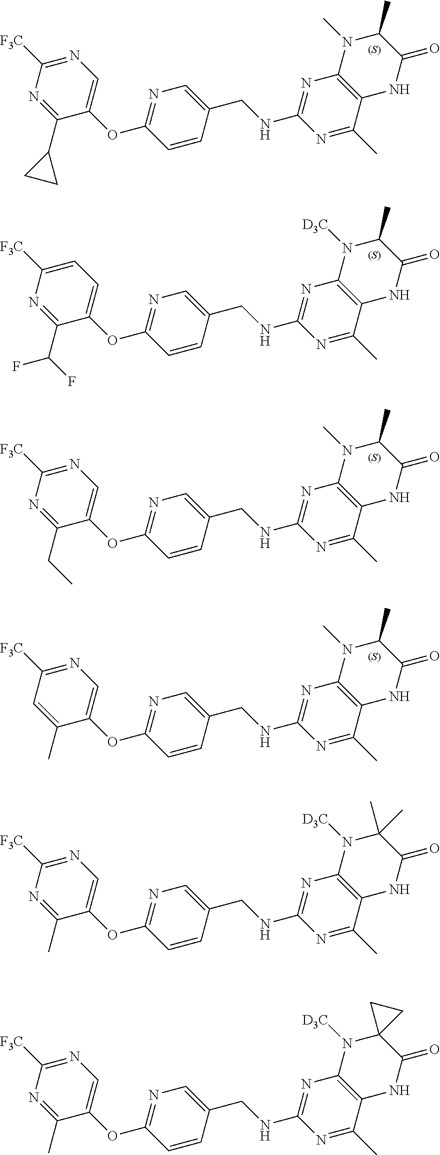 [chemical structure]
